(19) United States
(12) Patent Application Publication
(10) Pub. No.: US 2019/0184609 A1
(43) Pub. Date: Jun. 20, 2019

(54) SYSTEM AND METHOD FOR MANUFACTURING MICRONEEDLE DEVICES

(71) Applicant: University Medical Pharmaceuticals Corp., Irvine, CA (US)

(72) Inventors: Raymond Joseph Francis, Irvine, CA (US); David Bardin, Oceanside, CA (US); Gregory Lee Hunt, Irvine, CA (US); Antti Juhani Tauriainen, Oulu (FI); Markku Juha Kalervo Valkama, Oulu (FI); Ralph Liedert, Espoo (FI); Jarkko Tapani Tuominen, Espoo (FI)

(21) Appl. No.: 16/283,616

(22) Filed: Feb. 22, 2019

Related U.S. Application Data
(63) Continuation of application No. 15/982,426, filed on May 17, 2018.
(60) Provisional application No. 62/507,656, filed on May 17, 2017.

(51) Int. Cl. B29C 33/38; B29C 43/56; B29C 43/36; A61M 37/00; B29C 33/40; B29C 33/42; B29C 43/02; B29C 35/08

(52) U.S. Cl. CPC B29C 33/3878; B29L 2031/756; B29C 43/36; A61M 37/0015; B29C 33/3857; B29C 33/40; B29C 33/42; B29C 43/021; B29C 35/0805; A61M 2037/0053; B29C 2035/0822; A61M 2037/0046; A61M 2037/0023; B29C 33/424; B29K 2995/0025; B29K 2883/00; B29C 43/56

(57) ABSTRACT

A method for manufacturing microneedle devices and systems and tools for implementing same. The method can include manufacturing a replica mold and forming a microneedle array via the replica mold. The replica mold can be manufactured by disposing replica mold material on a master mold and curing the replica mold material. To reduce manufacturing time, the replica mold material preferably is cured at a high temperature for a relatively short time and then cooled quickly before removal from the master mold. The microneedle array can be formed by disposing microneedle material on the replica mold under vacuum and drying the microneedle material in single or successive disposing and drying operations. One or more optional backing layers can be added to the microneedle array when forming the microneedle device. Advantageously, the disclosed methods, systems and tools can be used to manufacture skin-applied patches for delivering cosmetic and therapeutic agents.

### CROSS-REFERENCE TO RELATED APPLICATIONS

This application is a continuation application of co-pending U.S. patent application Ser. No. 15/982,426, filed on May 17, 2018, which claims the benefit of, and priority to, U.S. Provisional Patent Application Ser. No. 62/507,656, filed May 17, 2017. Priority to the above-identified patent applications is expressly claimed, and the disclosures of these applications are hereby incorporated herein by reference in their entireties and for all purposes.

### FIELD

The disclosed embodiments relate generally to manufacturing systems and processes and more particularly, but not exclusively, to tools and processes for manufacturing microneedle devices, including skin-applied patches for delivering cosmetic and therapeutic agents to the skin.

### BACKGROUND

Microneedle arrays are used as transdermal and intradermal drug/therapeutic-delivery systems and to deliver polymers directly into and through the skin for cosmetic applications. Biodegradable microneedles are commonly used. Existing devices provide the biodegradable microneedles attached to a patch having a substrate layer that contacts the skin. In use, the substrate layer or patch is applied to the skin and pressure is applied which causes the microneedles to pierce the stratum corneum. One disadvantage of these devices is that the patch must remain affixed to the skin while the microneedles dissolve within the underlying skin layers. Microneedle dissolution may take several hours to a day or more, depending upon the specific microneedle composition. It is often inconvenient, unsightly, and/or uncomfortable for the user to wear the device for this extended period of time.

Microneedle arrays also are difficult to manufacture, particularly in mass production. The material forming the microneedle arrays typically is very viscous and presents challenges when shaped into the small form factor of microneedles. In addition, individual microneedles may not be fully formed during the manufacturing process and can be damaged during post-production handling.

In view of the foregoing, there is a need to efficiently manufacture and provide a biocompatible/biodegradable microneedle device that can effectively deliver the microneedles across the stratum corneum and be removed within a short period of time without affecting the performance of the device/microneedles.

### BRIEF DESCRIPTION OF DRAWINGS

FIG. 1A is an exemplary detail diagram illustrating an embodiment of a microneedle.

FIG. 1B is an exemplary detail diagram illustrating an alternative embodiment of the microneedle of FIG. 1A, wherein the microneedle has a diamond shape.

FIG. 2A is an exemplary top-level block diagram illustrating an embodiment of a microneedle device, wherein the microneedle device includes a plurality of the microneedles of FIG. 1A.

FIG. 2B is an exemplary plan view of the microneedle device of FIG. 2A, wherein the microneedles are arranged in a predetermined pattern.

FIG. 3A is an exemplary top-level block diagram illustrating an alternative embodiment of the microneedle device of FIGS. 2A-B, wherein the microneedles are physically connected via a residual layer of microneedle material.

FIG. 3B is an exemplary top-level block diagram illustrating another alternative embodiment of the microneedle device of FIGS. 2A-B, wherein the microneedles are disposed on an optional backing layer.

FIG. 4 is an exemplary top-level flow diagram illustrating an embodiment of a method for manufacturing the microneedle device of FIGS. 2A-B via a replica mold.

FIG. 5A is an exemplary detail diagram illustrating an embodiment of the replica mold of FIG. 4, wherein the replica mold defines a plurality of microneedle wells.

FIG. 5B is an exemplary plan view of the replica mold of FIG. 5A, wherein the microneedle wells are arranged in a predetermined pattern.

FIG. 6 is an exemplary top-level flow diagram illustrating an embodiment of a method for manufacturing the replica mold of FIGS. 5A-B via a master mold.

FIG. 7A is an exemplary top-level block diagram illustrating an embodiment of the master mold of FIG. 6, wherein the master mold includes a plurality of microneedle projections.

FIG. 7B is an exemplary plan view of the master mold of FIG. 7A, wherein the microneedle projections are arranged in a predetermined pattern.

FIG. 8A is an exemplary top-level block diagram illustrating an embodiment of the master mold of FIGS. 7A-B, wherein replica mold material is disposed on the master mold.

FIG. 8B is an exemplary top-level block diagram illustrating an alternative embodiment of the master mold of FIGS. 7A-B, wherein the replica mold material receives the microneedle projections and is cured on the master mold to form the replica mold.

FIG. 9A is an exemplary top-level block diagram illustrating an embodiment of the master mold of FIG. 8B, wherein the master mold is disposed on a cooling device for cooling the replica mold material of the replica mold.

FIG. 9B is an exemplary top-level block diagram illustrating an alternative embodiment of the cooling device of FIG. 9A, wherein the cooling device comprise a plurality of cooling devices.

FIG. 9C is an exemplary top-level block diagram illustrating another alternative embodiment of the cooling device of FIG. 9A, wherein the cooling device includes a plurality of cooling regions.

FIG. 10A is an exemplary top-level flow diagram illustrating an alternative embodiment of the method of FIG. 6, wherein the method includes manufacturing the master mold.

FIG. 10B is an exemplary top-level flow diagram illustrating another alternative embodiment of the method of FIG. 6, wherein the method includes separating the replica mold from the master mold.

FIG. 11 is an exemplary top-level flow diagram illustrating an embodiment of a method for forming the microneedle array of FIGS. 2A-B via the replica mold of FIGS. 5A-B.

FIG. 12 is an exemplary top-level block diagram illustrating an embodiment of the replica mold of FIGS. 5A-B, wherein microneedle material is disposed on the replica mold.

FIG. 13 is an exemplary top-level flow diagram illustrating an alternative embodiment of the method of FIG. 11, wherein the microneedle material is distributed into one or more microneedle wells formed within the replica mold.

FIG. 14A is an exemplary top-level block diagram illustrating an alternative embodiment of the replica mold of FIG. 12, wherein the microneedle material is distributed within the microneedle wells and is dried on the replica mold to form the microneedle array.

FIG. 14B is an exemplary top-level block diagram illustrating another alternative embodiment of the replica mold of FIG. 12, wherein the replica mold is disposed on a vacuum system for distributing the microneedle material into the microneedle wells.

FIG. 15 is an exemplary top-level flow diagram illustrating another alternative embodiment of the method of FIG. 11, wherein the method includes separating the microneedle array from the replica mold.

FIG. 16 is an exemplary top-level flow diagram illustrating an alternative embodiment of the method for forming the microneedle array of FIG. 11, wherein the microneedle material is disposed on the replica mold via a reservoir system.

FIG. 17 is an exemplary top-level detail diagram illustrating an embodiment of the reservoir system of FIG. 16.

FIG. 18A is an exemplary top-level block diagram illustrating an alternative embodiment of the replica mold of FIG. 14B, wherein the microneedle material is received by, and/or stored in, the reservoir system of FIG. 17.

FIG. 18B is an exemplary top-level block diagram illustrating an alternative embodiment of the replica mold of FIG. 18A, wherein the replica mold cooperates with the reservoir system.

FIG. 18C is an exemplary top-level block diagram illustrating an alternative embodiment of the replica mold of FIG. 18B, wherein the reservoir system begins to dispense the microneedle material onto the replica mold.

FIG. 18D is an exemplary top-level block diagram illustrating an alternative embodiment of the replica mold of FIG. 18C, wherein the reservoir system stops dispensing the microneedle material onto the replica mold.

FIG. 18E is an exemplary top-level block diagram illustrating an alternative embodiment of the replica mold of FIG. 18D, wherein the replica mold and the reservoir system are separated.

FIG. 19A is an exemplary top-level block diagram illustrating another alternative embodiment of the replica mold of FIG. 14B, wherein the reservoir system of FIG. 17 is disposed within a vacuum chamber.

FIG. 19B is an exemplary top-level block diagram illustrating an alternative embodiment of the replica mold of FIG. 19A, wherein the vacuum chamber is in a sealed position.

FIG. 19C is an exemplary top-level block diagram illustrating an alternative embodiment of the replica mold of FIG. 19B, wherein a vacuum system for applying a vacuum to the vacuum chamber is enabled.

FIG. 19D is an exemplary top-level block diagram illustrating an alternative embodiment of the replica mold of FIG. 19C, wherein the reservoir system is disposed adjacent to the replica mold.

FIG. 19E is an exemplary top-level block diagram illustrating an alternative embodiment of the replica mold of FIG. 19D, wherein the vacuum applied by the vacuum system is adjusted.

FIG. 19F is an exemplary top-level block diagram illustrating an alternative embodiment of the replica mold of FIG. 19E, wherein a shutter system of the reservoir system enters an open position for enabling reservoir openings formed by the reservoir system to communicate with microneedle wells formed in the replica mold.

FIG. 19G is an exemplary top-level block diagram illustrating an embodiment of the shutter system of FIG. 19F, wherein the shutter system is disposed in a closed position.

FIG. 19H is an exemplary top-level block diagram illustrating an alternative embodiment of the shutter system of FIG. 19F, wherein the shutter system is disposed in the open position.

FIG. 19I is an exemplary top-level block diagram illustrating an alternative embodiment of the replica mold of FIG. 19F, wherein the reservoir system dispenses the microneedle material onto the replica mold.

FIG. 19J is an exemplary top-level block diagram illustrating an alternative embodiment of the replica mold of FIG. 19I, wherein the shutter system is disposed in the closed position.

FIG. 19K is an exemplary top-level block diagram illustrating an alternative embodiment of the replica mold of FIG. 19J, wherein the replica mold is disposed distally from the vacuum system.

FIG. 19L is an exemplary top-level block diagram illustrating an alternative embodiment of the replica mold of FIG. 19K, wherein the vacuum system is disabled.

FIG. 20 is an exemplary top-level flow diagram illustrating an alternative embodiment of the method for forming the microneedle array of FIG. 16, wherein the reservoir system is disposed within the vacuum chamber of FIGS. 19A-L.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

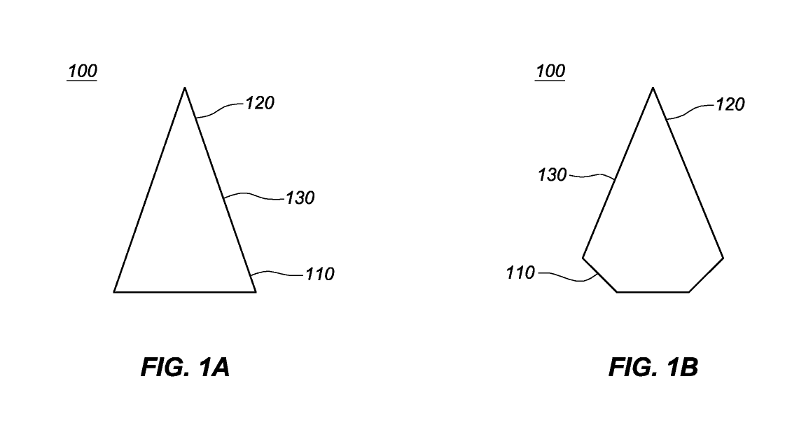

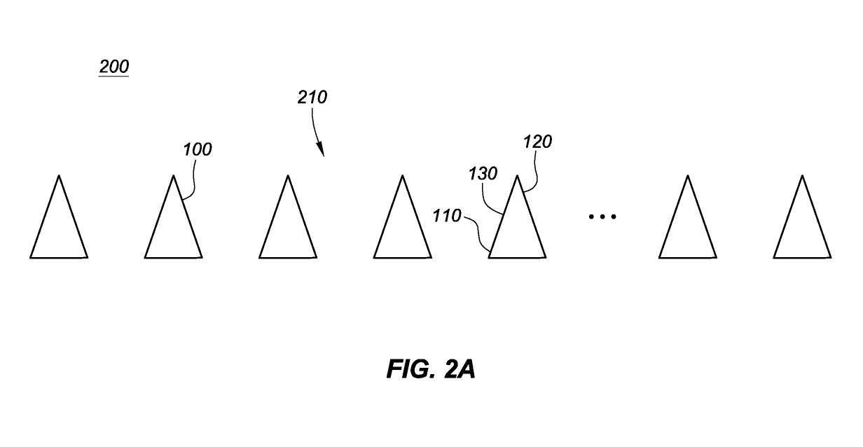

Since currently-available microneedle devices must remain affixed to the skin while the microneedles dissolve and are inconvenient, unsightly, and/or uncomfortable for the user, a microneedle device that overcomes these disadvantages can prove desirable and provide a basis for a wide range of microneedle device applications, such as delivery of polymeric and/or biocompatible compositions beneath and/or within the skin surface to reduce (or eliminate) fine lines, wrinkles, stretch marks, scars, cellulite, and other skin imperfections or to smooth, texture, tighten, and/or hydrate the skin. This result can be achieved, according to one embodiment disclosed herein, through the manufacture of a microneedle 100 as illustrated in FIGS. 1A-B.

Turning to FIGS. 1A-B, the microneedle 100 can be provided as a three-dimensional structure with a predetermined shape, size and/or dimension and can comprise a preselected microneedle material 130. The microneedle 100 can include a base region 110 and an apex (or upper body) region 120 that is opposite the base (or lower body) region 110. A cross-section of the microneedle 100 adjacent to the base region 110 preferably is less than a cross-section of the microneedle 100 adjacent to the apex region 120. Stated somewhat differently, the cross-section of the microneedle 100 can generally decrease from the base region 110 to the apex region 120. The microneedle 100, for example, can have a generally conical shape as illustrated in FIG. 1A, a generally diamond shape as shown in FIG. 1B, or a generally pyramidal shape.

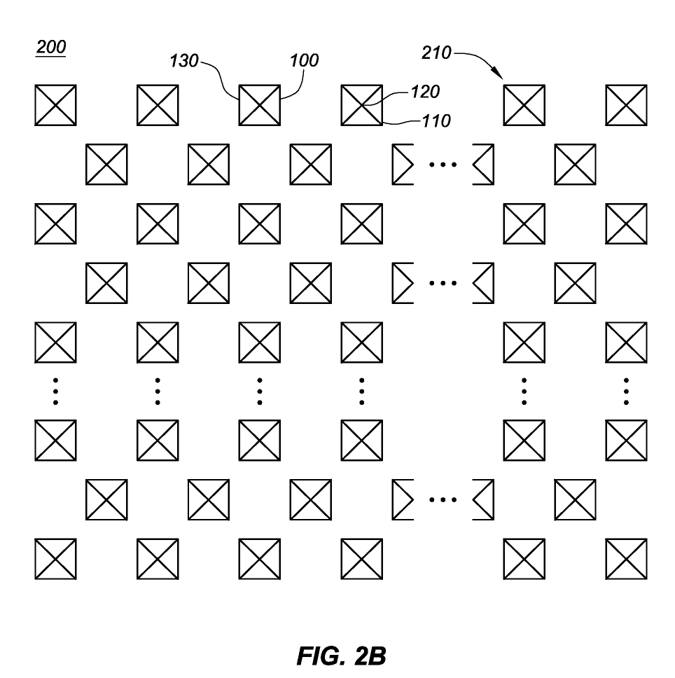

A selected microneedle 100 can be characterized by an apex region 120 and a base region 110 that may or may not be symmetrical. The base region 110 may have any convenient dimension including for example, less than 5%, 10%, 15%, 20%, 25%, 30%, 35%, 40%, 45%, or 50% of a total height of the microneedle 100. Advantageously, if provided with a diamond shape and/or a pyramidal shape, the microneedle 100 may more rapidly detach from a microneedle device, such as the microneedle device 200 shown in FIGS. 2A-B, because the base region 110 provides a small point of attachment between the microneedle 110 and the microneedle device.

The microneedles 100 may have any height suitable to application to the skin. The microneedle height may be selected to reach or target specific depths or skin layers including for example, the epidermis, dermis, and subcutaneous tissue, or specific boundary regions such as the dermal/epidermal junction.

In one embodiment, the preselected microneedle material 130 can comprise any polymeric and/or nonpolymeric solution suitable for making microneedles for an intended purpose. Exemplary polymeric solutions can include a natural or synthetic polymeric solution, including a sugar, a sugar alcohol, a polysaccharide, a carbohydrate, cellulose, and/or a starch. In some embodiments, the microneedles 100 can be intended to deliver cosmetic and therapeutic agents to and across the skin by pressing the relatively hard microneedles of the array into the skin surface such that the microneedles penetrate the stratum corneum, epidermis, and/or dermis. Suitable polymeric solutions or ingredients that may be used in the manufacture of microneedles include, but are not limited to, gelatin, hydroxypropyl methylcellulose (HPMC), ethanol, arginine, polyols, silk, superabsorbent hydrogels, superporous hydrogels, polymethyl vinyl ether-alt-maleic anhydride (PMVE/MA), maltose, HEPES (influenza vaccine stabilizer), glycerol, collagen, calcium hydroxylapatite, poly-L-lactic acid (PLLA), polymethyl methacrylate (PMMA), alginate, fructose, raffinose, chondroitin sulfate, galactose, dextrin, self-assembling peptides, etc.

In some embodiments, the microneedles 100 contain at least one polymer selected from the group consisting of pullulan, hyaluronic acid (HA), polylactic acid (PLA), polyglycolic acid (PGA), poly(lactic-co-glycolic acid) (PLGA), cellulose, sodium carboxymethyl cellulose (SCMC), hydroxyethyl cellulose (HEC), hydroxypropyl cellulose (HPC), hydroxypropyl methylcellulose (HPMC), amylopectin (AMP), silicone, polyvinylpyrolidone (PVP), polyvinyl alcohol (PVA), poly(vinylpyrrolidone-co-methacrylic acid) (PVA-MAA), polyhydroxyethylmethacrylate (pHMEA), polyethlene glycol (PEG), polyethylene oxide (PEO), polyacrylic acid, chrondroitin sulfate, dextrin, dextran, maltodextrin, chitin, chitosan, mono- and polysaccharides, galactose, and maltose. In particular embodiments, the microneedles 100 comprise hyaluronic acid or a mixture of hyaluronic acid and pullulan. In some embodiments, the microneedles 100 also contain at least one sugar alcohol (e.g., mannitol, sorbitol, and xylitol). In some embodiments, the microneedles 100 also contain an active ingredient.

In some embodiments, the microneedles 100 contain 1.0%-7.5% hyaluronic acid (HA), 2.5%-15% pullulan, and 0.5%-5.0% mannitol. The HA may be crosslinked or uncrosslinked. Optionally, uncrosslinked HA may be present at about 3%-6%. Optionally, crosslinked HA may be present at about 1%-4%. Optionally, pullulan is present in a concentration of about 3%-12%, including 3%-6%, 5%-10%, and 4%-12%.

In other embodiments, the microneedles 100 contain a mixture of low molecular weight HA (“low MW HA”) and high molecular weight HA (“high MW HA”). In some embodiments, the low MW HA is present in a concentration of about 0.25-5%, including for example, 1.0-3.0% (e.g., about 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, and 5%) and the high MW HA is present in a concentration of about 0.25%-3.0%, including for example, about 0.25%, 0.5%, 0.75%, 1.0%, 1.25%, 1.5%, 1.75%, 2.0%, 2.25%, 2.5%, 2.75%, and 3.0%.

By “microneedles” is meant a plurality of protrusions, as described herein, and have a height, measured from the inner surface of the intermediate layer, or the inner surface of the substrate layer, if present, to the tip of the microneedle, of about 100 μm-1,500 μm, including for example about 300 μm-1,000 μm, or about 400 μm-800 μm, including about 100 μm, 200 μm, 300 μm, 400 μm, 500 μm, 600 μm, 700 μm, 800 μm, 900 μm, 1,000 μm, 1,100 μm, 1,200 μm, 1,300 μm, 1,400 μm, and 1,500 μm. In other embodiments, the aspect ratio (i.e., ratio of height to base) of the microneedles 100 is about 1.0-4.0, including about 1.5-3.5, and 2.0-3.0, including, for example, about 1.0, 1.25, 1.5, 1.75, 2.0, 2.25, 2.5, 2.75, 3.0, 3.25, 3.5, 3.75, and 4.0. In some embodiments, the microneedles 100 have absolute dimension for the base of about 50 μm, 100 μm, 150 μm, 200 μm, 250 μm, 300 μm, 350 μm, 400 μm, 450 μm, 500 μm, 550 μm, or 600 μm. In other embodiments, the microneedles 100 have an absolute dimension (height to base) of about 400:200 μm, 600:300 μm, or 800:400 μm. Microneedles 100 may be formed into any suitable shape including, for example, conical, diamond, tetrahedral, and pyramidal shapes.

By “pullulan” is meant a polysaccharide polymer consisting of maltotriose units in which the three glucose units in maltotriose are joined by an α-1,4 glycosidic bond and consecutive maltotriose units are joined to each other by an α-1,6 glycosidic bond. In some embodiments, pullulan has an average molecular weight of about 5,000-20,000 Da, including about 7,500-15,000 Da (e.g., about 5,000, 6,000, 7,000, 8,000, 9,000, 10,000, 11,000, 12,000, 13,000, 14,000, 15,000, 16,000, 17,000, 18,000, 19,000, and 20,000 Da, or more).

When referring to relative polymer concentrations (i.e., percentages), for convenience, reference is made to the polymer concentration in solution prior to molding and drying.

Advantageously, one or more microneedles 100 can collectively comprise a microneedle device 200 as illustrated in FIGS. 2A-B. The microneedle device 200, in other words, can include a microneedle array 210 having at least one microneedle 100. The microneedles 100 in the microneedle array 210 can be disposed in any predetermined arrangement and/or configuration. As shown in FIG. 2A, for example, the microneedles 100 can be uniformly aligned such that the base region 110 of a selected microneedle 100 is positioned adjacent to the base region 110 of a neighboring microneedle 100. The apex regions 120 of the microneedles 100 can extend in substantially the same direction. Although shown and described as having microneedles 100 with uniform shape, size and/or dimension for purposes of illustration only, the microneedle array 210 can include microneedles 100 with uniform and/or different shapes, sizes and/or dimensions as desired.

FIG. 2B is an exemplary plan view of the microneedle device 200 with the apex regions 120 of the microneedles 100 shown as extending from the drawing sheet. The microneedles 100 can be arranged in any predetermined pattern. For example, the microneedle array 210 can include one or more microneedles 100 disposed in a regularly-distributed pattern and/or one or more microneedles 100 disposed in an irregularly-distributed (or random) pattern. An exemplary regularly-distributed pattern for the microneedle array 210 can comprise can include a plurality of parallel rows of the microneedles 100 and/or a plurality of parallel columns of the microneedles 100. As shown in FIG. 2B, for instance, the microneedles 100 can be arranged in offset (or out-of-phase) rows. Incorporated by reference U.S. patent application Ser. No. 15/821,314, filed on Nov. 22, 2017, also sets forth additional detail about the structure and application of the microneedle device 200.

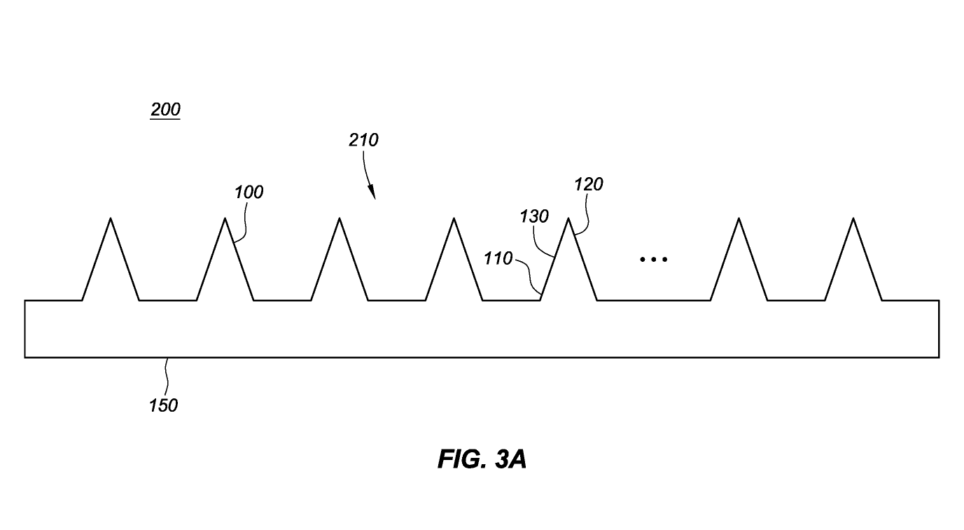

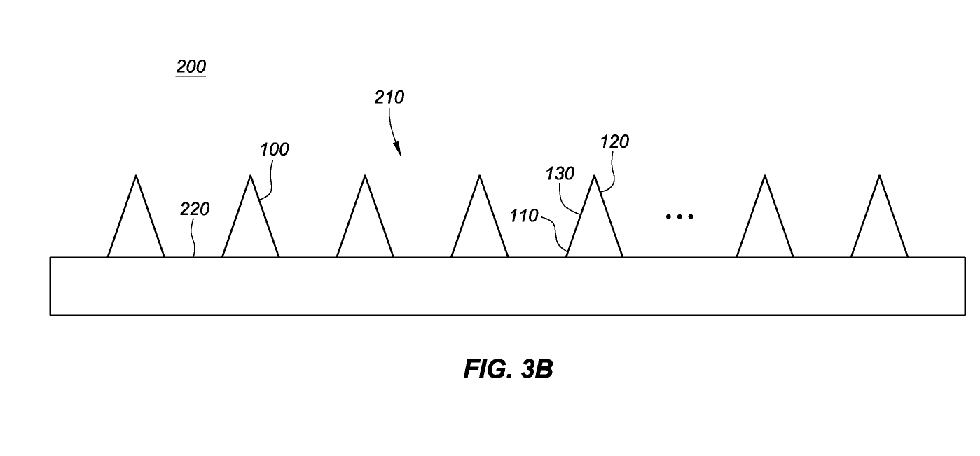

The microneedles 100 of the microneedle array 210 can comprise at least one individual (or separate) microneedle 100 and/or at least one group of cooperating (or coupled) microneedles 100. Stated somewhat differently, the microneedle array 210 can comprise one or more microneedles 100 that are discontinuous as illustrated in FIG. 2A and/or one or more microneedles 100 that are physically connected as illustrated in FIGS. 3A-B. The cooperating microneedles 100 can be coupled in any conventional manner. For example, the cooperating microneedles 100 are shown in FIG. 3A as being coupled via the preselected microneedle material 130. The preselected microneedle material 130 can extend from, and couple, the base regions 110 of the cooperating microneedles 100 in one embodiment. In other words, the cooperating microneedles 100 can be physically connected via a residual layer (or sheet) 150 of the preselected microneedle material 130. The cooperating microneedles 100 thereby can comprise a contiguous structure.

By comprising a contiguous structure, the cooperating microneedles 100 advantageously are easier to manufacture than individual microneedles. In addition, the residual layer 150 of the preselected microneedle material 130 can contain the active ingredients of the preselected microneedle material 130 that may be delivered into the skin through diffusion upon dissolution of the microneedles 100. A dosage of the active ingredients delivered by the microneedle array 210 thereby can be increased. Furthermore, the cooperating microneedles 100 as a contiguous structure can make a backing layer 220 (shown in FIG. 3B) optional, for example, when the microneedle material 130 forms a residual layer 150 with some strength and/or flexibility.

Additionally and/or alternatively, the microneedle device 200 is illustrated in FIG. 3B as including the optional backing layer 220. The cooperating microneedles 100 of the microneedle array 210 thereby can be coupled via the backing layer 220. By coupling the microneedles 100 in the microneedle array 210, the predetermined arrangement, configuration and/or pattern of the microneedles 100 advantageously can be maintained. Incorporated by reference U.S. patent application Ser. No. 15/821,314, filed on Nov. 22, 2017, also sets forth additional detail about the coupling of the microneedles 100, including the backing layer 220, as well as optional treatment of the backing layer 220 with a liquid, such as water, after applying the microneedle device 200 to the skin during use.

Manufacture of the Microneedle Device 200

The microneedle device 200 can be manufactured via a manufacturing method as set forth herein, such as wet etching or dry etching using a silicon base, precision machining using metal or resin (electro-discharge machining, laser processing, grinding, hot embossing, injection molding, etc.), and/or machinery cutting, without limitation. For embodiments in which hollow microneedles 100 are desired, the microneedles 100 can be hollowed during the molding process and/or by secondary processing, such as via laser cutting.

Other suitable methods for manufacturing the microneedle array 210 can include centrifuge casting (see, for example, U.S. Patent Application Publication No. 2009/0182306 to Lee, et al.) and lithography (see, for example, Moga, et al., “Rapidly-Dissolvable Microneedle Patches Via a Highly Scalable and Reproducible Soft Lithography Approach,” Adv. Mater. 2013; DOI:10.102/adma.201300526). In centrifuge casting, a microneedle mold (not shown) that defines one or more microneedle mold cavities is produced by an appropriate technique such as photolithography or by etching in a silicon substrate, such as a substrate formed from polydimethyl siloxane (PDMS). An aqueous polymeric solution can be prepared and placed into the microneedle mold as, for example, a viscous and/or elastic gel or a non-viscous solution. The filled microneedle mold can be centrifuged under conditions that promote filling of the microneedle mold cavities. The filled microneedle mold can be dried. Optionally, the microneedle mold can be partially filled several times with the same and/or different polymeric solutions to allow for customization of the microneedles 100 over their length and/or for the incorporation of active ingredients in specific portions/layers of the microneedles 100. In other casting techniques, the polymer solution can be forced into the microneedle mold using positive pressure (rollers, e.g. the Particle Replication in Non-wetting Templates (or PRINT) process) or negative pressure (or a vacuum).

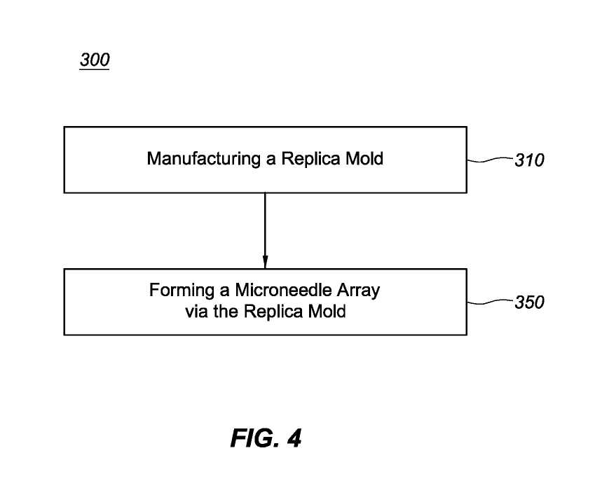

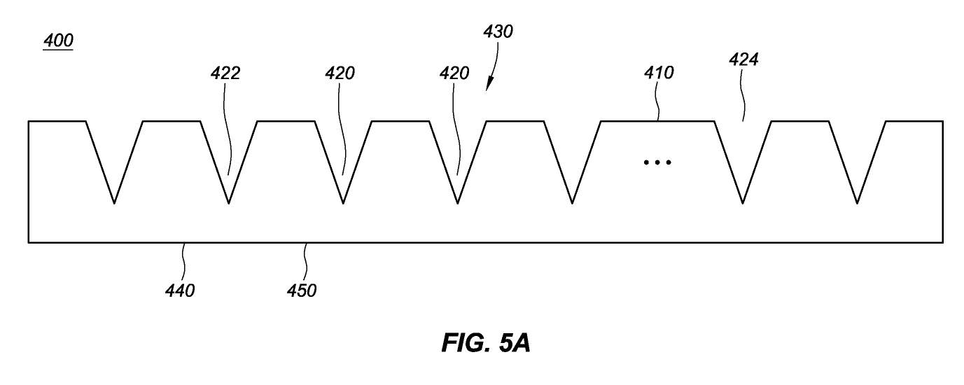

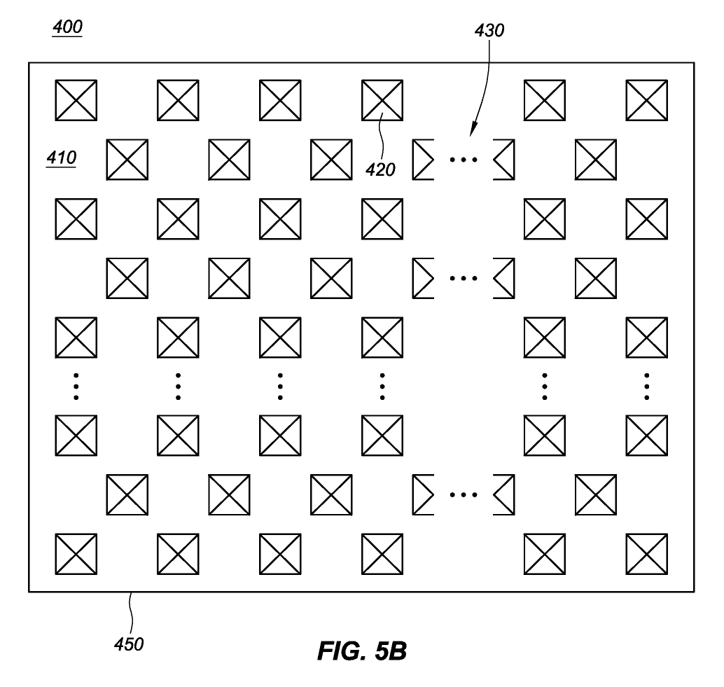

An exemplary method 300 for manufacturing the microneedle device 200 is shown in FIG. 4. As shown in FIG. 4, the method 300 includes manufacturing a replica mold 400 (shown in FIGS. 5A-B), at 310, and forming a microneedle array 210 (shown in FIGS. 2A-B) via the replica mold 400, at 350. Since a selected replica mold 400 can be reusable in some embodiments, manufacturing the replica mold 400, at 310, can be considered to be optional for manufacturing the microneedle device 200, at 350. In other words, the selected replica mold 400 can be repeatedly used to successively form multiple microneedle arrays 210, at 350, such that a new replica mold 400 need not be manufactured for the microneedle array 210 of each microneedle device 200.

Manufacture of the Replica Mold 400

An exemplary replica mold 400 is illustrated in FIGS. 5A-B. Turning to FIGS. 5A-B, the replica mold 400 can be provided as a three-dimensional structure with a predetermined shape, size and/or dimension and can comprise a preselected replica mold material 450. The replica mold 400 can include an upper region 410 and a lower region 440. The upper region 410 preferably is opposite the lower region 440.

The replica mold 400 can define a plurality of microneedle wells 420. Each of the microneedle wells 420 comprises a respective recess 422 that is formed within the replica mold 400 and that communicates with an associated opening 424 formed in the upper region 410. The microneedle wells 420 preferably are provided as a microneedle well array 430 that corresponds with the microneedles 100 in the microneedle array 210 (collectively shown in FIGS. 2A-B). Stated somewhat differently, each microneedle well 420 preferably has a shape, size and/or dimension that is a negative (or inverse) of the shape, size and/or dimension of a corresponding microneedle 100 in the microneedle array 210. Thereby, when filled with the microneedle material 130, a selected microneedle well 420 molds the microneedle material 130 into the shape, size and/or dimension of the corresponding microneedle 100.

FIG. 5B is an exemplary plan view of the replica mold 400 with the microneedle wells 420 of the microneedle well array 430 shown as extending into the drawing sheet. The microneedle wells 420 can be arranged in any predetermined pattern. For example, the microneedle well array 430 can include one or more microneedle wells 420 disposed in a regularly-distributed pattern and/or one or more microneedle wells 420 disposed in an irregularly-distributed (or random) pattern. An exemplary regularly-distributed pattern for the microneedle well array 430 can comprise can include a plurality of parallel rows of the microneedle wells 420 and/or a plurality of parallel columns of the microneedle wells 420. As shown in FIG. 5B, for instance, the microneedle wells 420 can be arranged in offset (or out-of-phase) rows, which correspond to the predetermined pattern of microneedles 100 illustrated in FIG. 2B. Although shown and described as having microneedle wells 420 with uniform shape, size and/or dimension for purposes of illustration only, the microneedle well array 430 can include microneedle wells 420 with uniform and/or different shapes, sizes and/or dimensions as desired.

In one embodiment, the replica mold material 450 can comprise any suitable silicone elastomer, such as PDMS. Preferred characteristics of the silicone elastomer include biocompatibility (such as medical grade or implantable class), low viscosity, fast curing rate, high gas permeability, low elongation (without being brittle), a predetermined mixing ratio (such as a predetermined base-to-curing agent mixing ratio between about 1:1 and 10:1) and/or compatibility with dispenser systems. More specifically, the silicone elastomer preferably has a viscosity between 1-5 Pas and/or a curing time between one and fifteen minutes when exposed to heat or ultraviolet light. Exemplary silicone elastomers can include: SYLGARD® 184 manufactured by Dow Corning Corporation of Auburn, Mich.; the Wacker Silpuran series of silicone elastomers manufactured by Wacker Chemie AG of Munich, Germany; MED-6015 or MED-6215 silicone elastomer manufactured by NuSil™ Technology LLC of Carpinteria, Calif.; and/or Bluesil ESA 7246 manufactured by Bluestar Silicones USA Corp. of Brunswick, N.J. In some embodiments, the silicone elastomer can be hydrophobic; whereas, in other embodiments, such as replica molds 400 for forming individual microneedles 100, the silicone elastomer can be hydrophilic. In one embodiment, the silicone elastomer can include one or more surface modifying agents. An exemplary surface modifying agent can be n-Wet 410D silicone compound manufactured by Enroute Interfaces, Inc., of Ontario, Canada.

Additionally and/or alternatively, the replica mold material 450 can comprise any suitable type of porous material. Exemplary suitable porous materials can include polyethylene oxide (PEO) polybutylene terephthalate (PBT) block copolymers, and sulfonated polyetheretherketon (SPEEK); and/or METAPOR® ceramic composite material manufactured by Portec Ltd. of Aadorf, Switzerland.

In one embodiment, the replica mold material 450 can comprise a translucent (or transparent) material. Use of the translucent replica mold material 450 to form the replica mold 400 can enable light-based curing, such as ultraviolet (UV) curing, of the microneedle material 130 within the microneedle wells 420. The translucent replica mold material 450 likewise can facilitate visualization and/or quality control as the microneedle wells 420 are filled with the microneedle material 130, during the molding process and/or after the microneedles are cured. An operator thereby can visually monitor the microneedle material 130 within the microneedle wells 420 during manufacture of the microneedle device 200 via camera and/or fluorescence quality analysis.

In a different embodiment, the replica mold material 450 can comprise a black (or opaque) material. Use of the black replica mold material 450 to form the replica mold 400 can enable rapid infrared (IR) curing of the microneedle material 130 within the microneedle wells 420. The black replica mold material 450 likewise can help to maintain heat within the replica mold 400.

The properties of silicone microneedle molds provide significant advantages over conventional molds that are made from hard plastic, such as acrylic, or conventional silicone molds that are fixed to a hard substrate, such as a plastic, a ceramic, or an epoxy. The flexibility of silicone rubber advantageously minimizes damage to the cured microneedles 100 when the microneedle array 210 is removed from the mold, especially when the mold is manually peeled and/or automatedly peeled, such as via a robot arm, from the cured microneedle array 210 rather than the microneedle array 210 being peeled from the replica mold 400.

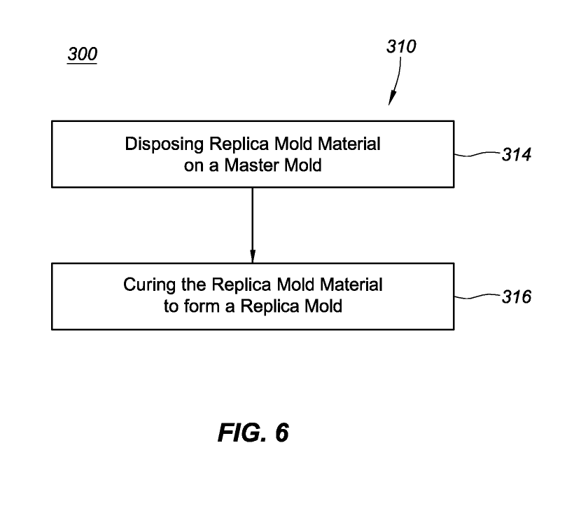

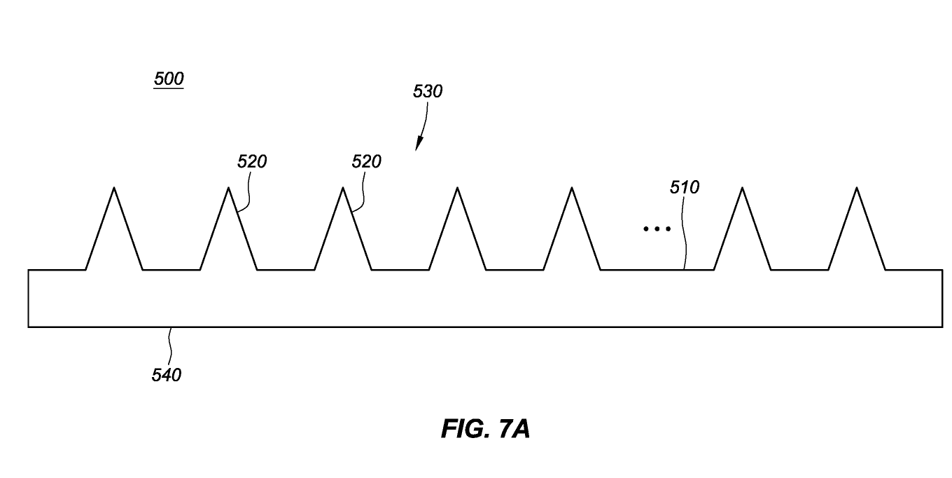

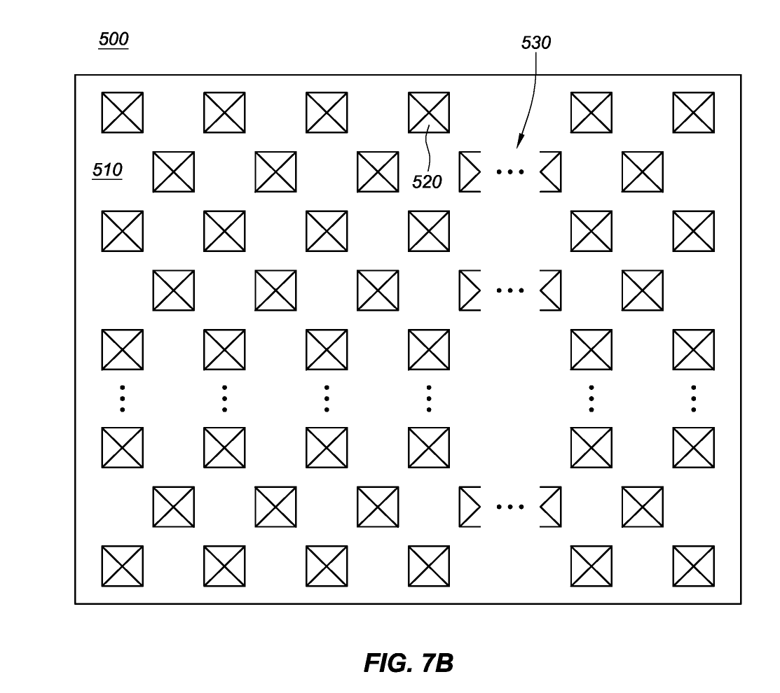

The replica mold 400 (shown in FIGS. 5A-B) can be manufactured via any suitable process. An exemplary method 310 for manufacturing the replica mold 400 is illustrated in FIG. 6. As shown in FIG. 6, the method 310 includes disposing replica mold material 450 on a master mold 500 (shown in FIGS. 7A-B), at 314, and curing the replica mold material 450 to form the replica mold 400, at 316. The master mold 500 can be manufactured from any suitable preselected master mold material, which preferably has a particularly high thermal conductivity, can be reliably micro-machined, is relatively lightweight and/or is relatively low cost. Preferably comprising a reusable mold, the master mold 500 can be created on a hard substrate such as a metal (e.g., aluminum, copper or brass), a plastic (e.g., an acrylic), silicon (e.g. SU-8 epoxy-type, near-UV photoresist, manufactured by MicroChem Inc. of Newton, Mass., on a silicon wafer), a ceramic and/or an epoxy, without limitation. In other words, a selected master mold 500 can be repeatedly used to successively manufacture multiple replica molds 400.

An exemplary master mold 500 is illustrated in FIGS. 7A-B. Turning to FIGS. 7A-B, the master mold 500 can be provided as a three-dimensional structure with a predetermined shape, size and/or dimension. The master mold 500 can include an upper region 510 and a lower region 540. The upper region 510 preferably is opposite the lower region 540.

The master mold 500 can define a plurality of microneedle projections 520. The microneedle projections 520 can extend from the upper region 510 and preferably are provided as a microneedle projection array 530 that corresponds with the microneedles 100 in the microneedle array 210 (collectively shown in FIGS. 2A-B). Stated somewhat differently, each microneedle projection 520 preferably is a replica of a corresponding microneedle 100 in the microneedle array 210 and has the same shape, size and/or dimension as the corresponding microneedle 100. In one embodiment, the master mold 500 can include one or more dividers (or walls) (not shown) that at least partially enclose the microneedle projections 520. The dividers advantageously can prevent the replica mold material 450 from spreading beyond the microneedle projections 520 and thereby reduce an amount of scrap when the replica mold material 450 is disposed on the master mold 500.

FIG. 7B is an exemplary plan view of the master mold 500 with the microneedle projections 520 of the microneedle projection array 530 shown as extending from the drawing sheet. The microneedle projections 520 can be arranged in any predetermined pattern. For example, the microneedle projection array 530 can include one or more microneedle projections 520 disposed in a regularly-distributed pattern and/or one or more microneedle projections 520 disposed in an irregularly-distributed (or random) pattern. An exemplary regularly-distributed pattern for the microneedle projection array 530 can comprise can include a plurality of parallel rows of the microneedle projections 520 and/or a plurality of parallel columns of the microneedle projections 520. As shown in FIG. 7B, for instance, the microneedle projections 520 can be arranged in offset (or out-of-phase) rows, which correspond to the predetermined pattern of microneedles 100 illustrated in FIG. 2B. Although shown and described as having microneedle projections 520 with uniform shape, size and/or dimension for purposes of illustration only, the microneedle projection array 530 can include microneedle projections 520 with uniform and/or different shapes, sizes and/or dimensions as desired.

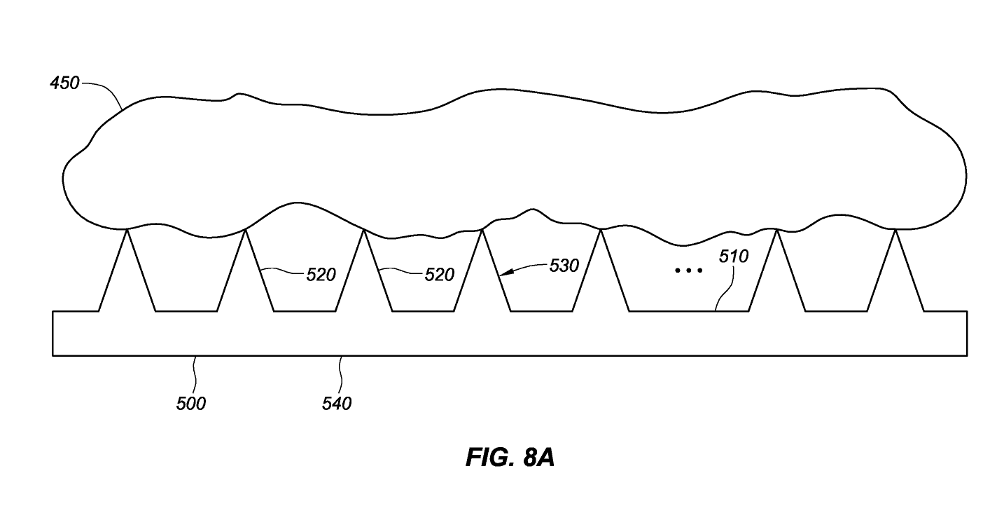

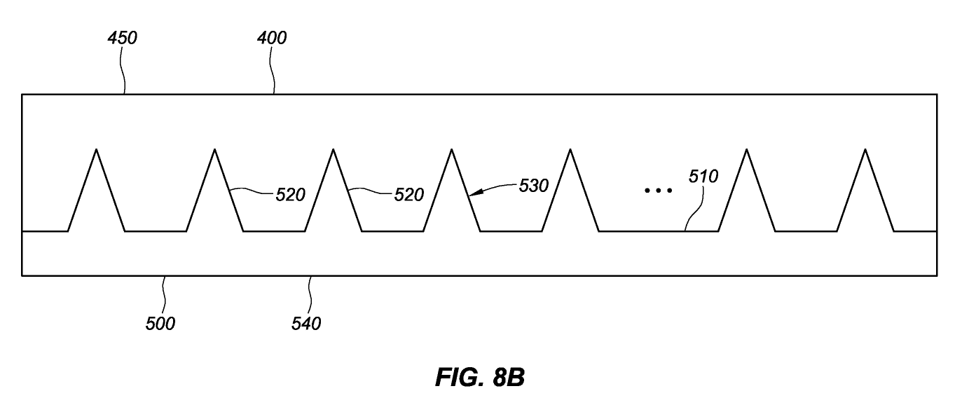

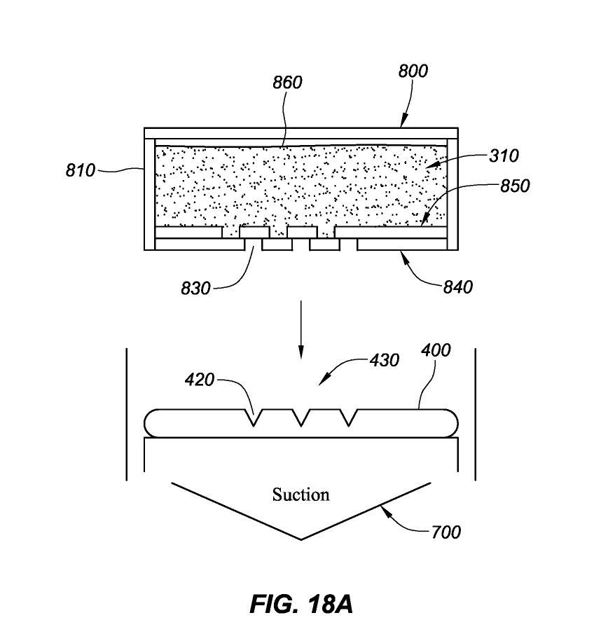

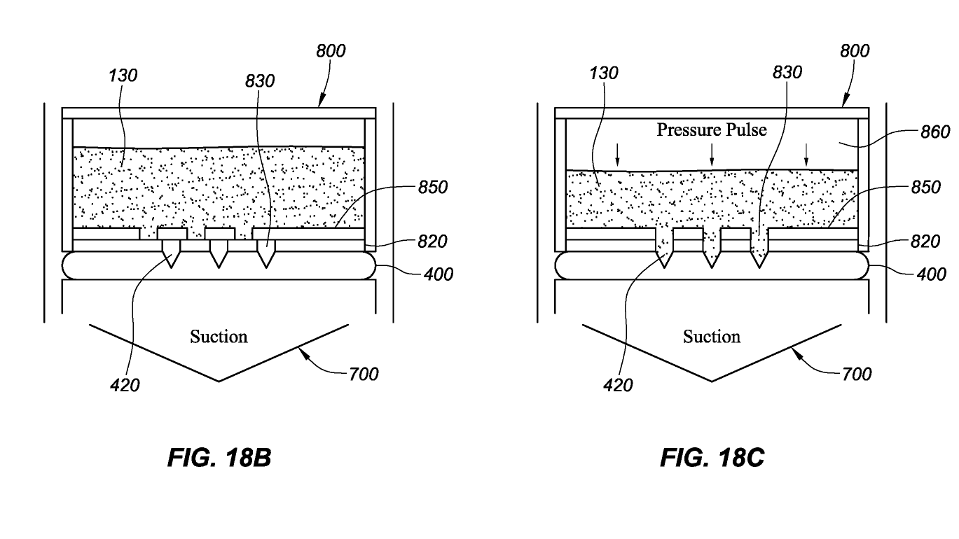

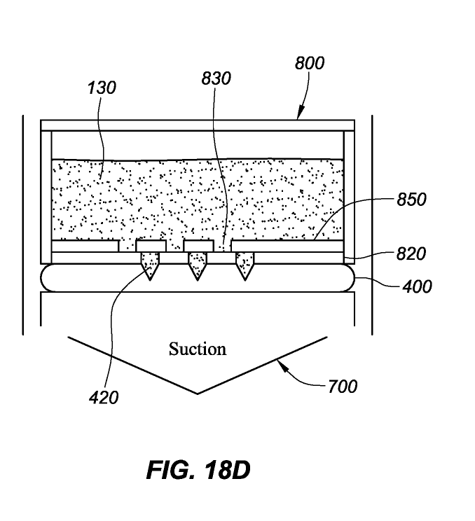

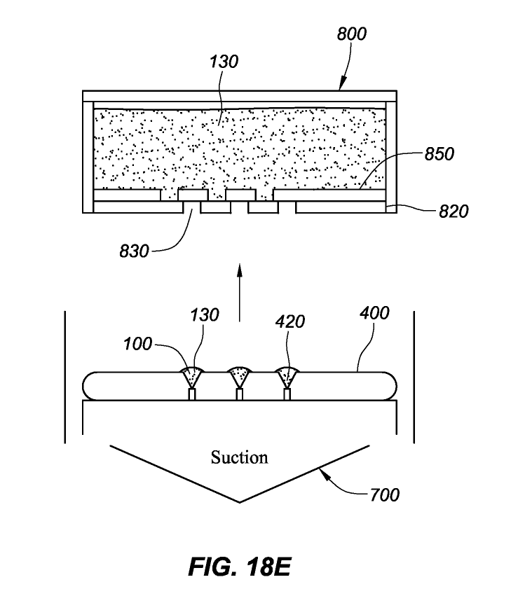

In the manner discussed above with reference to FIG. 6, the replica mold material 450 can be disposed on the master mold 500, at 314. FIG. 8A shows the master mold 500 with the replica mold material 450 being disposed thereon. The replica mold material 450 can be disposed on the master mold 500 in a manual manner, such as via a syringe, and/or in an automated manner, such as via a dispenser nozzle. If the replica mold material 450 comprises a silicone elastomer, such as PDMS material, for example, the PDMS material can be mixed and/or poured under preselected environmental conditions. Exemplary environmental conditions can include a clean room with a predetermined clean room temperature, such as 21° C.±1° C., and/or a predetermined clean room relative humidity, such as 40% RH±10% RH. The PDMS material can be mixed in a manual manner and/or in an automated manner and/or can be degassed via a vacuum system 700 (shown in FIGS. 18A-E). In one embodiment, the PDMS material can be degassed for a predetermined time period, such as fifteen minutes, at the clean room temperature. Preferably, the PDMS material can be degassed in a periodic (or cyclic) manner with the PDMS material being subjected to a vacuum during a first time period, such as one minute, and then being permitted to return to normal pressure and settle during a second time period, such as three minutes. The first and second time periods can be repeated, as desired.

The replica mold material 450 is dispensed onto the master mold 500 to a suitable height and can receive the microneedle projections 520 as shown in FIG. 8B. The dispensed replica mold material 450 can be cured, at 316, to form the desired replica mold 400 with the microneedle well array 430 of the microneedle wells 420 as shown in FIGS. 5A-B.

The replica mold material 450 may be cured using any suitable curing process, such as heat or UV, including according to the instructions of a manufacturer of the replica mold material 450. In the case of PDMS material, the typical curing process involves the application of moderate heat (about 60-100° C.) for an extended period of time (about 35-90 minutes). The moderate heat can be applied to the PDMS material in any conventional manner, such as by placing the lower region 540 of the filled master mold 500 on a heat source (not shown), such as a hot plate or an oven.

The extended curing times associated with the manufacture of the replica mold 400 can be limiting in high-throughput operations, including those operations in which the resulting microneedle device 200 will be stored and/or shipped in the replica mold 400. Thus, in some embodiments, the replica mold 400 may have only a single use. In an effort to increase the production rate of the replica mold 400, various PDMS curing conditions were evaluated in order to reduce the curing time of the replica mold 400 without significantly impairing the performance of the cured PDMS material in the microneedle molding process.

It was surprisingly discovered that microneedle molding performance was not impaired when the PDMS material was cured at a high temperature (e.g., at least 150° C., 160° C., 170° C., 180° C., 190° C., 200° C. or more) for a relatively short time (e.g., less than five minutes, less than ten minutes, or less than fifteen minutes) and cooled quickly before removal of the master mold 500. In other words, the PDMS material can be cured at a preselected temperature (and/or within a preselected range of temperatures) for a preselected time period (and/or within a preselected range of time periods). Exemplary preselected temperature ranges can include a predetermined temperature range between 150° C. and 300° C., including any temperature sub-ranges, such as a five degree sub-range (i.e., between 180° C. and 185° C.) and/or a ten degree sub-range (i.e., between 180° C. and 190° C.), within the predetermined temperature range, without limitation. Exemplary preselected time period ranges can include a predetermined time period range between one minute and twenty minutes, including any time period sub-ranges, such as a one minute sub-range (i.e., between nine minutes and ten minutes) and/or a five minute sub-range (i.e., between five minutes and ten minutes), within the preselected time period range, without limitation. When micro-molding, PDMS material generally is not cured at these elevated temperatures (and shorter curing times) because the resulting product may be more brittle and/or may contain crystallized regions. As the thermosetting PDMS material hardens with a higher modulus, the PDMS material may be less suitable for certain applications than that created by lower temperature cures.

The production rate of replica molds 400 can be limited by an extended time to heat the replica mold material. In an effort to reduce manufacturing time for replica molds 400, a selected master mold 500 can include a predetermined number of the microneedle projection arrays 530 such that each of the microneedle projection arrays 530 can manufacture a separate replica mold 400. The predetermined number of the replica molds 400 can be manufactured concurrently via the selected master mold 500. Additionally and/or alternatively, more than one master mold 500 can be disposed on, and heated by, a selected heat source at a given time and/or a plurality of heat sources can be provided to heat a plurality of the master molds 500.

The production rate of replica molds 400 likewise can be limited by an extended time to cool the replica mold material. For example, contrary to conventional cooling methods, rapid and controlled cooling of the PDMS material can be achieved by moving the master mold 500 from the heat source onto a cool surface to quickly dissipate the heat from the master mold 500 and thus from the PDMS material disposed on the master mold 500. In one embodiment, the cooling time for the PDMS material can be reduced via water cooling. The master mold 500, for example, can define one or more internal and/or external cooling channels (not shown) and an automatic water circulation cooling system (not shown) can circulate water through the cooling channels of the master mold 500, cooling the master mold 500 and thus the PDMS material on the master mold 500.

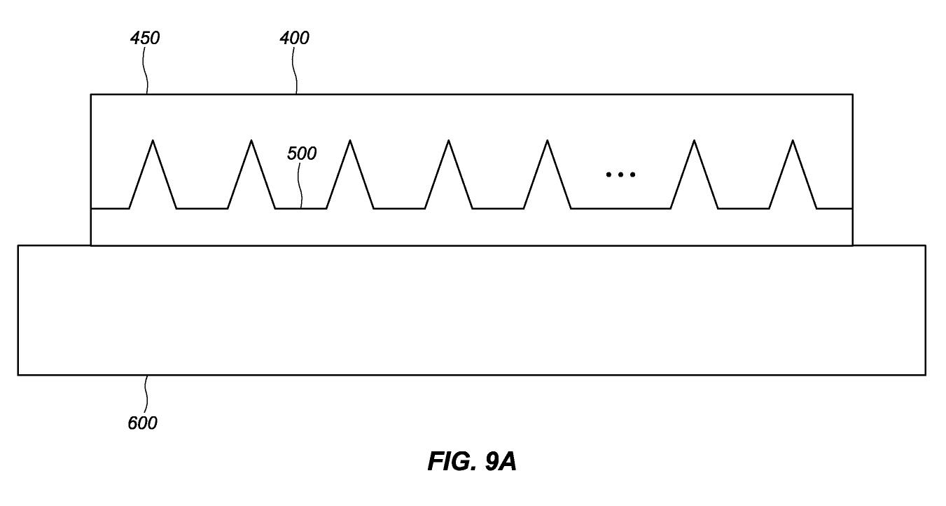

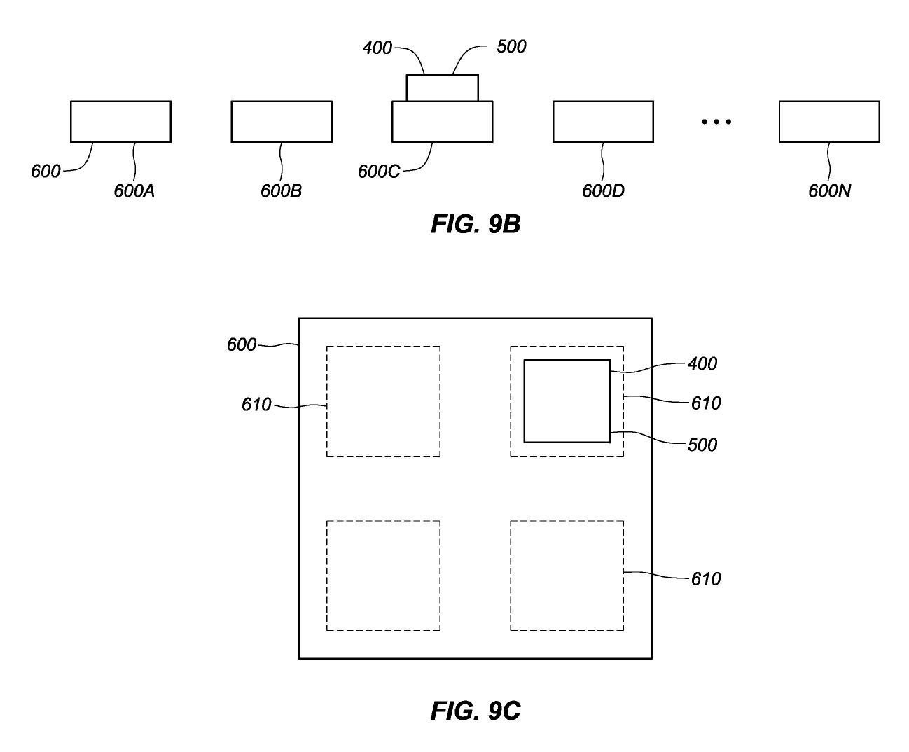

Turning to FIG. 9A, the master mold 500 with the replica mold material 450 is shown as being disposed on a cooling device for cooling the replica mold material 450 to form the replica mold 400. The cooling device can comprise any type of suitable cooling device. For example, the cooling device can comprise a metal block 600 at an ambient room temperature, such as 21° C. The material used to form the metal block 600 preferably has a particularly high thermal conductivity, is relatively lightweight and/or is relatively low cost such as aluminum, copper or brass. The heated master mold 500 with the replica mold material 450 can be disposed on the metal block 600 for a predetermined time period (and/or within a preselected range of time periods). Exemplary preselected time period ranges can include a predetermined time period range between thirty seconds and ten minutes, including any time period sub-ranges, such as a one minute sub-range (i.e., between four minutes and five minutes) and/or a three minute sub-range (i.e., between two minutes and five minutes), within the preselected time period range, without limitation. The heated master mold 500 with the replica mold material 450 thereby can be cooled to a preselected reduced temperature (and/or a preselected reduced temperature range), such as between 30° C. and 35° C. within the predetermined time period (and/or within the preselected range of time periods).

In one embodiment, the cooling device can comprise a plurality of cooling devices. For example, FIG. 9B shows that the cooling device can comprise a predetermined number of the metal blocks 600. The heated master mold 500 with the replica mold material 450 can be disposed on a first metal block 600A for the predetermined time period to be cooled to a first reduced temperature. Upon expiry of the predetermined time period, the heated master mold 500 with the replica mold material 450 can be disposed on a second metal block 600B for the predetermined time period to be cooled to a second reduced temperature. The heated master mold 500 with the replica mold material 450 then can be disposed on a third metal block 600C for the predetermined time period to be cooled to a third reduced temperature and so on until the heated master mold 500 with the replica mold material 450 achieves the preselected reduced temperature (and/or a preselected reduced temperature range). The metal blocks 600 can comprise a linear series of metal blocks 600A-600N, as shown in FIG. 9B, and/or can comprise a looped series of metal blocks 600A-600N, wherein the heated master mold 500 with the replica mold material 450 returns to metal block 600A after being cooled on metal block 600N. Movement and/or positioning of the heated master mold 500 with the replica mold material 450 on the metal blocks 600 can be performed in a manual manner and/or in an automated manner, such as via a pick-and-place machine.

Additionally and/or alternatively, the cooling device can include a plurality of cooling regions. The cooling device of FIG. 9C is shown as being a metal block 600 with a plurality of cooling regions 610. The heated master mold 500 with the replica mold material 450 can be disposed on a selected cooling region 610 for the predetermined time period to be cooled to a first reduced temperature. Upon expiry of the predetermined time period, the heated master mold 500 with the replica mold material 450 can be disposed on a different cooling region 610 for the predetermined time period to be cooled to a second reduced temperature. The heated master mold 500 with the replica mold material 450 then can be disposed on another cooling region 610 for the predetermined time period to be cooled to a third reduced temperature and so on until the heated master mold 500 with the replica mold material 450 achieves the preselected reduced temperature (and/or a preselected reduced temperature range). Movement and/or positioning of the heated master mold 500 with the replica mold material 450 on the cooling regions 610 can be performed in a manual manner and/or in an automated manner, such as via a pick-and-place machine.

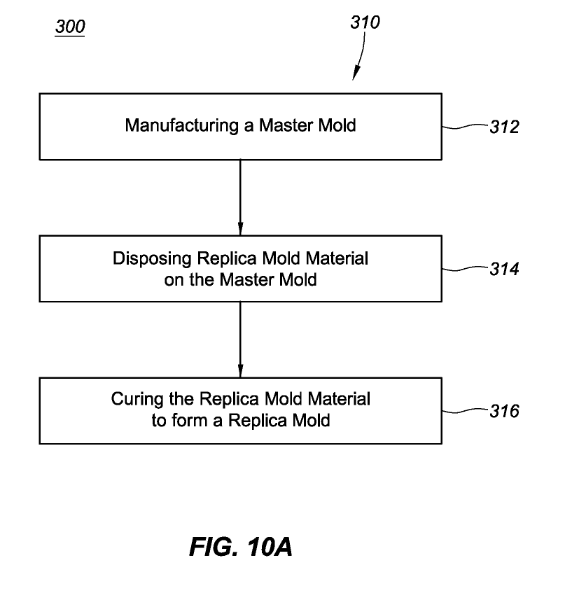

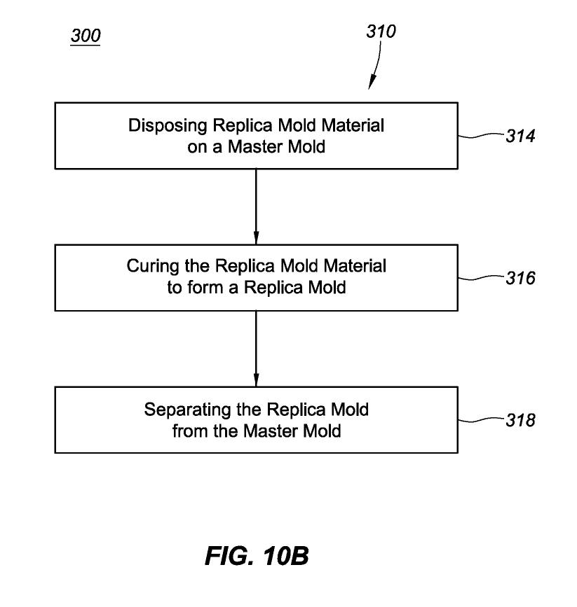

After curing of the replica mold material 450 is complete, the replica mold 400 optionally can be separated from the master mold 500, at 318, as shown in FIG. 10B. The replica mold material 450 preferably is easy to release from the master mold 500. In other words, the replica mold 400 should be removable from the master mold 500 without incurring damage to either the replica mold 400, the master mold 500 or both. The resultant negative replica mold 400 can be suitable for use in forming the microneedle array 210 of the microneedle device 200, at 350 (shown in FIG. 4).

Manufacture of the Microneedle Array 210

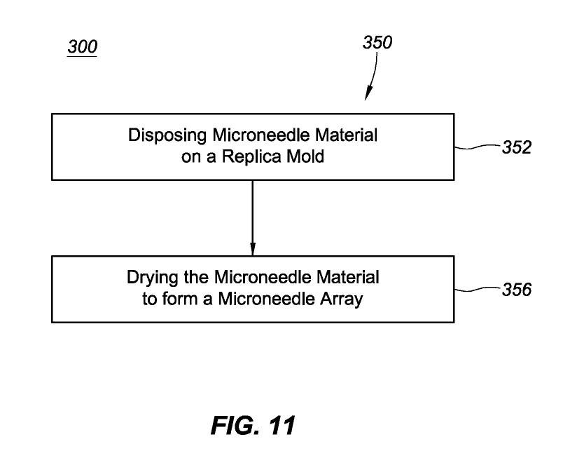

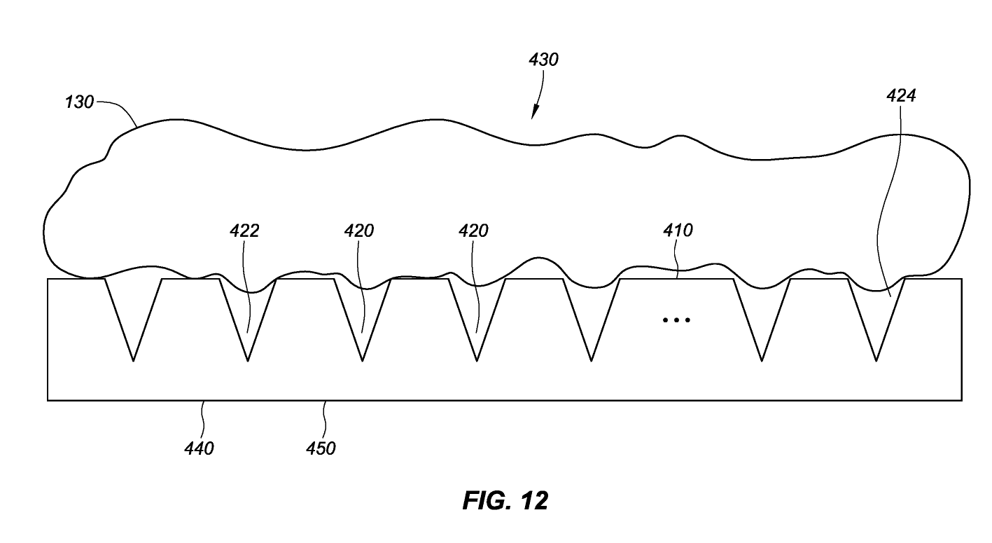

In one embodiment, the microneedle array 210 can be formed, at 350, in the manner illustrated in FIG. 11. As shown in FIG. 11, the microneedle array 210 can be formed, at 350, by disposing the microneedle material 130 on the replica mold 400 (shown in FIG. 12), at 352, and drying (and/or curing) the microneedle material 130 as disposed on the replica mold 400, at 356, to form the microneedle array 210 (shown in FIGS. 2A-B, 3A). The replica mold 400 may be retained in a carrier jig (i.e., a rigid reservoir) (not shown) to facilitate filling, handling, and/or other processes associated with the manufacture of the microneedle array 210. In the manner discussed in more detail above, the microneedles 100 can comprise cooperating microneedles 100 that can be physically connected via a residual layer 150 of the preselected microneedle material 130 in the manner illustrated in FIG. 3A and/or separate microneedles 100 in the manner illustrated in FIG. 2A.

Manufacture of the Microneedle Array 210 with a Residual Layer 150

As set forth in additional detail above with reference to FIG. 3A, the microneedles 100 can be physically connected via a residual layer 150 of the preselected microneedle material 130. To form the microneedles 100, the microneedle material 130 can be disposed on the replica mold 400. Stated somewhat differently, the replica mold 400 can be coated with an excess of uncured microneedle material 130. In one embodiment, the microneedle material 130 can be dispensed in droplets to one or more predetermined positions on the replica mold 400 to help assure that optimal coverage of the replica mold 400 can be achieved.

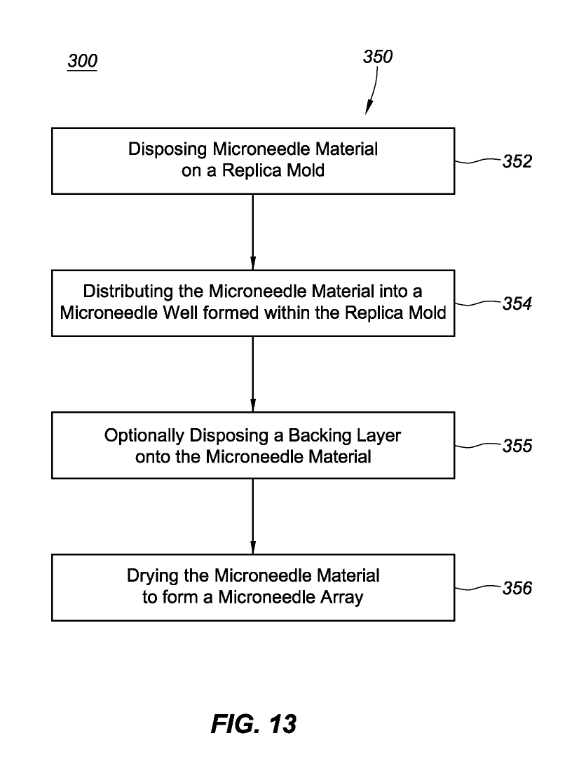

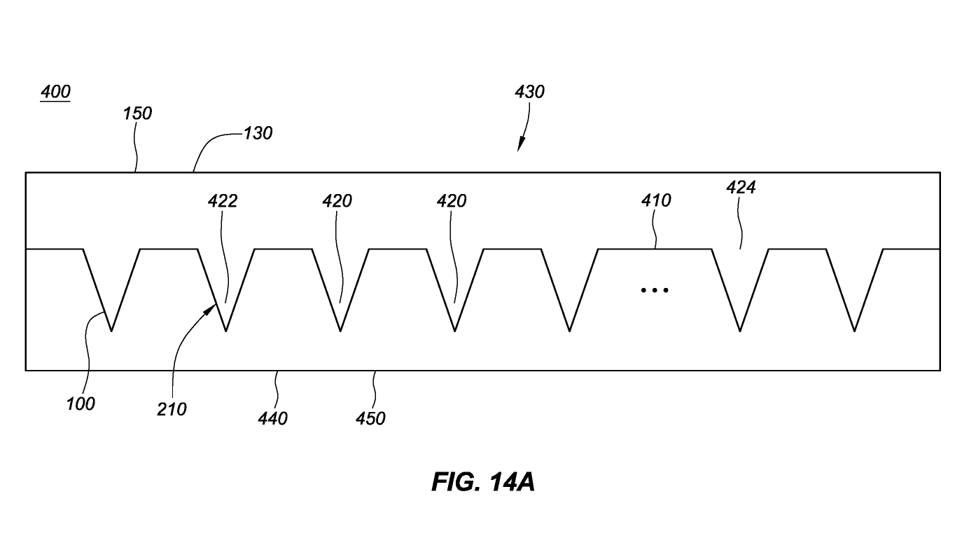

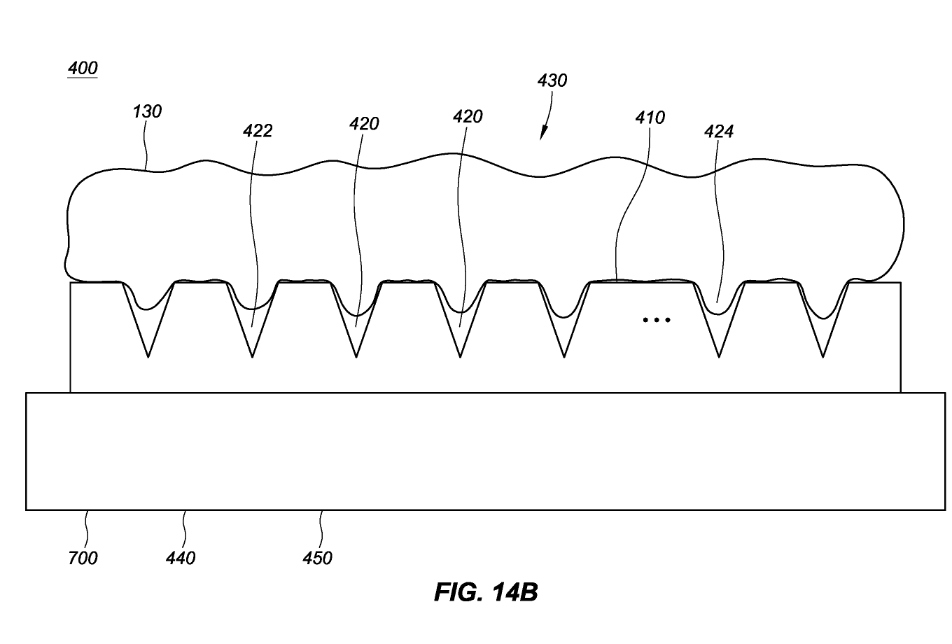

Turning to FIGS. 13 and 14A-B, forming the microneedle array 210, at 350, can include distributing, at 354, the microneedle material 130 into one or more microneedle wells 420 formed in the replica mold 400. The microneedle material 130 preferably fills each of the microneedle wells 420 from the opening 424 formed in the upper region 410 of the replica mold 400 to the recess 422 that is formed within the replica mold 400. The microneedle material 130, when cured, thereby can form microneedles 100 each having the base region 110 and the apex region 120 in the manner described above with reference to FIGS. 1A-B.

Advantageously, manufacturing the individual needles 100 in this manner can generate less scrap than conventional manufacturing methods, such as a standard coating process. Scrap can be reduced, for example, because the individual needles 100 can be manufactured without the residual layer 150 of microneedle material 130. Furthermore, the manufacture of the individual microneedles 100 in the manner set forth above can present cost savings such as when the active ingredient(s) of the preselected microneedle material 130 are expensive. Exemplary microneedle materials 130 with high-cost active ingredients can include, but are not limited to, crosslinked hyaluronic acid as well as certain drugs, vaccines, toxins, etc.

The microneedle material 130 can be distributed evenly across the replica mold 400 in any suitable manner. Exemplary suitable manners for distributing the microneedle material 130 can include passive distribution via, for instance, a flowing action of the microneedle material 130 and/or active distribution via positive pressure. The positive pressure can be applied to the microneedle material 130 in any appropriate manner, including via mechanical compression such as a flat sheet (formed from metal and/or plastic), a weight, a roller, a stencil, a squeegee or other similar tool.

As illustrated in FIG. 13, forming the microneedle array 210, at 350, optionally can include disposing backing material, such as the backing layer 220 (shown in FIG. 3B), onto the microneedle material 130, at 355. The backing layer 220 can comprise one or more additional layers of different materials that can be placed on the microneedle material 130 for facilitating efficient bonding between the various layers that comprise the final microneedle device 200 (shown in FIGS. 2A-B, 3A-B). The backing layer 220 can be placed on the microneedle material 130 as a part of the disposing of the microneedle material 130 on the replica mold 400, at 352, the distributing the microneedle material 130 into one or more microneedle wells 420, at 354, and/or the drying the microneedle material 130, at 356, and/or can comprise a separate (or independent) placement process. A suitable backing layer 220 can include, for example, a dissolvable layer (e.g., comprising pullulan or another water soluble polymer or polysaccharide), an air- and/or liquid-permeable mesh, an occlusive layer, a non-occlusive layer and/or any other type of backing layer.

In one embodiment, the backing layer 220 is non-occlusive, water-permeable, and adapted to support the microneedle material 130 and/or the additional layers of different materials that can be placed on the microneedle material 130. Backing layer 220 may be formed from any suitable web, mesh, or woven material including, for example, pressed, woven and non-woven cellulose fibers, PLA webs, and membrane filters (e.g., porous films of polyester, nylon, and the like). The backing layer 220 may be substantially the same dimension the microneedle material 130 and/or the additional layers and/or may overhang the microneedle material 130 and/or the additional layers in one or dimension. In one embodiment, the backing layer 220 can have an overhang region that extends beyond the dimension of the microneedle material 130 and/or the additional layers. The overhang region may or may not be water-permeable and may be made from the same or different material than the remainder of the backing layer 220 that overlays the microneedle material 130 and/or the additional layers.

Advantageously, the backing layer 220 can include an optional adhesive (not shown), such as a pressure-sensitive adhesive, a medical grade adhesive, and/or a skin-friendly adhesive. For selected applications of the microneedle device 200, such as microneedle devices 200 intended for being affixed to the skin of a user, the adhesive can be disposed on a skin-facing region of the backing layer 220.

In the manner set forth above, a flat sheet (not shown) can be placed on top of the microneedle material 130 and any backing layer(s) 220. In one preferred embodiment, the flat sheet can have a preselected shape, size and/or dimension that is greater than the predetermined shape, size and/or dimension of the microneedle array 210 and preferably can be at least partially disposed and/or retained within a carrier jig (not shown). The flat sheet can form a substantially gas-tight seal with the carrier jig. In another embodiment, a gas-impermeable top layer (not shown) can be placed on top of the microneedle material 130 and any backing layer(s) 220. The gas-impermeable top layer can cover the microneedle array 210 and/or an inner dimension of the carrier jig. The gas-impermeable top layer may be incorporated into the microneedle device 200 as a part of the backing layer 220 and/or may be disposable prior to use of the microneedle device 200. For example, the gas-impermeable top layer can be retained after the microneedle material 130 has been dried, at 356 (shown in FIG. 13) and/or throughout subsequent storage of the microneedle device 200 as a protective layer, a water-impermeable layer, and/or an occlusive backing layer of the microneedle device 200.

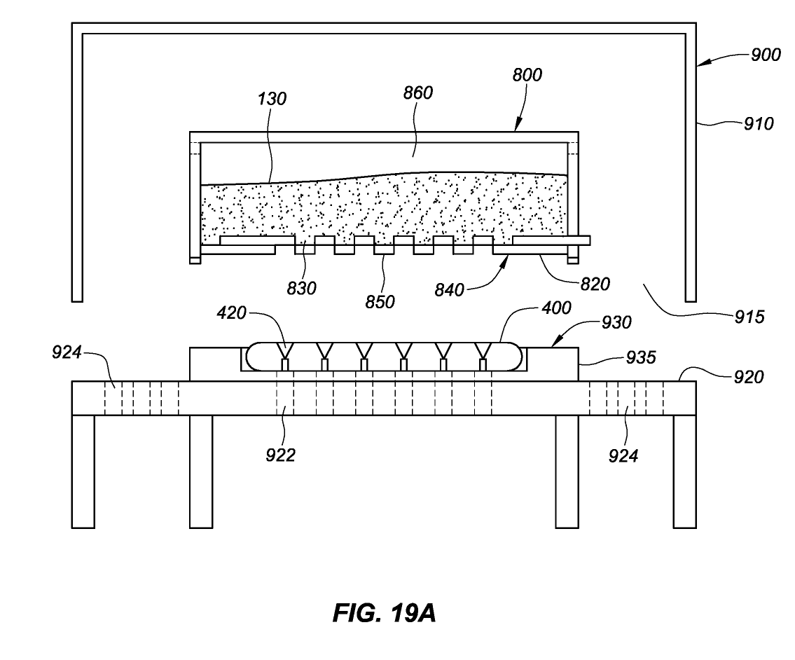

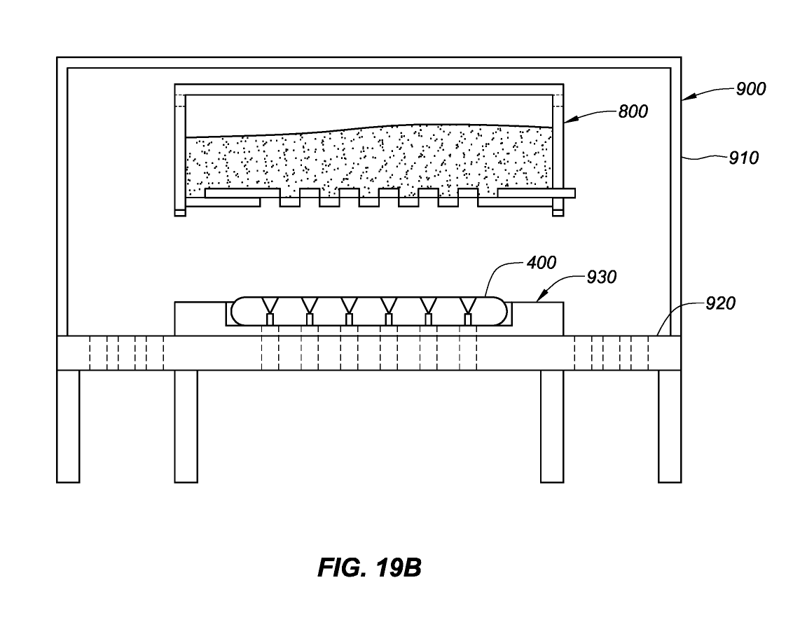

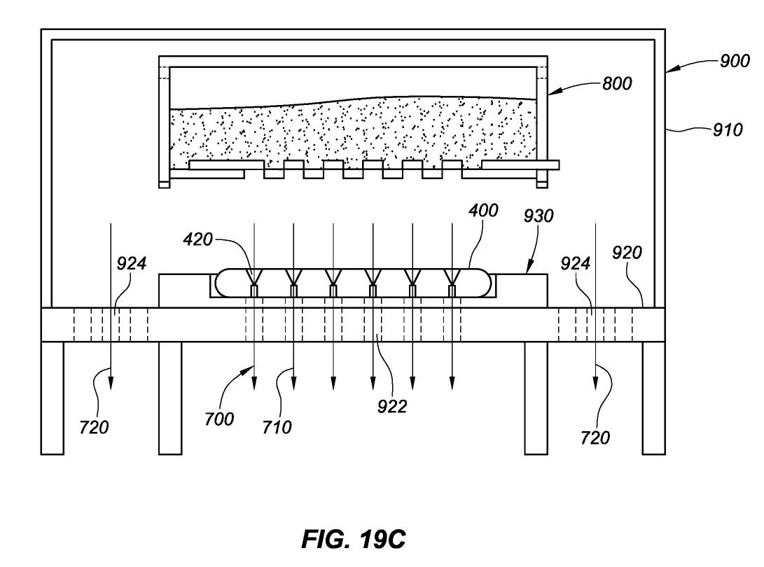

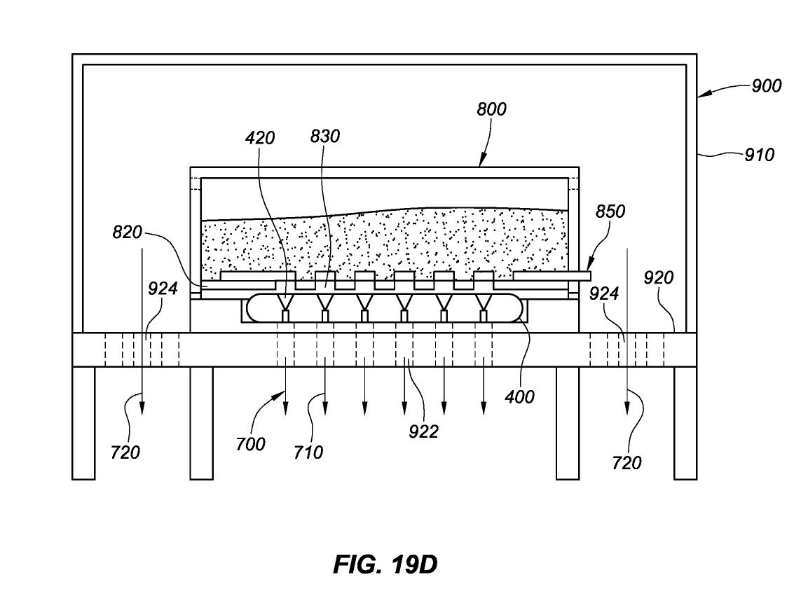

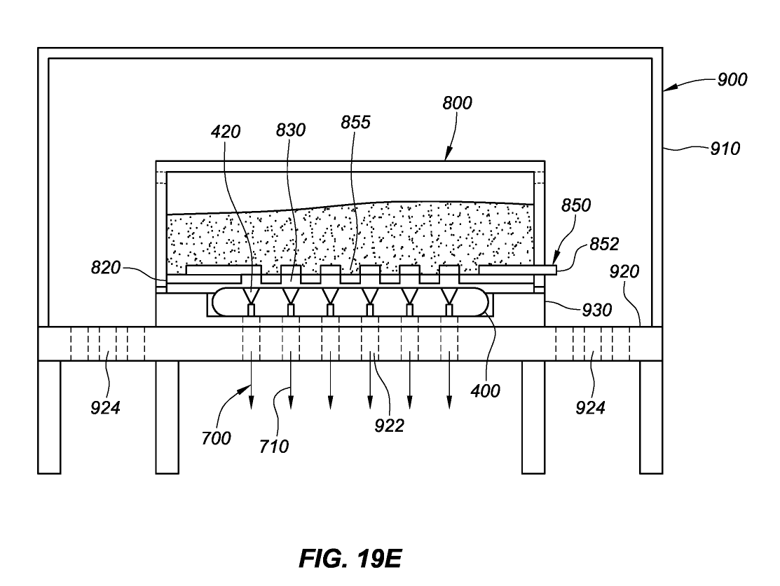

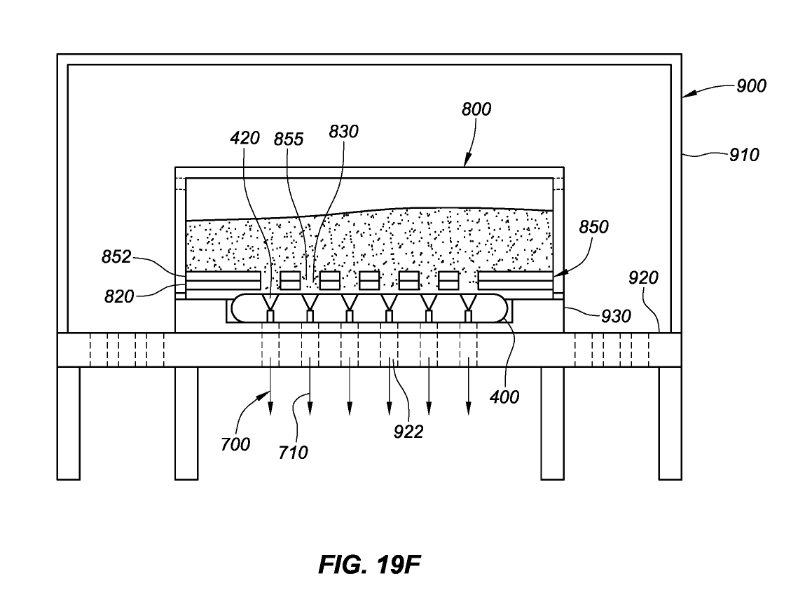

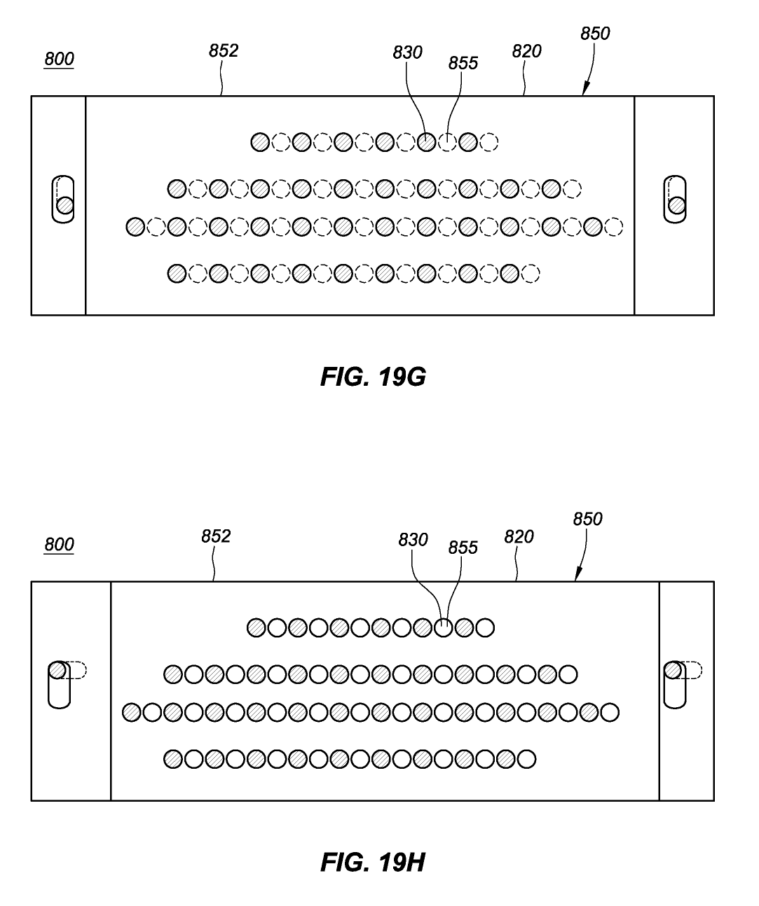

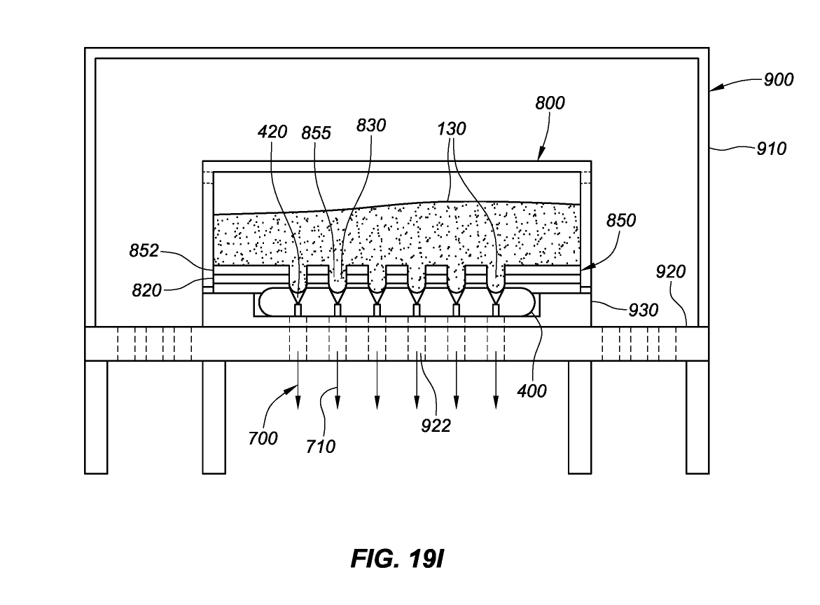

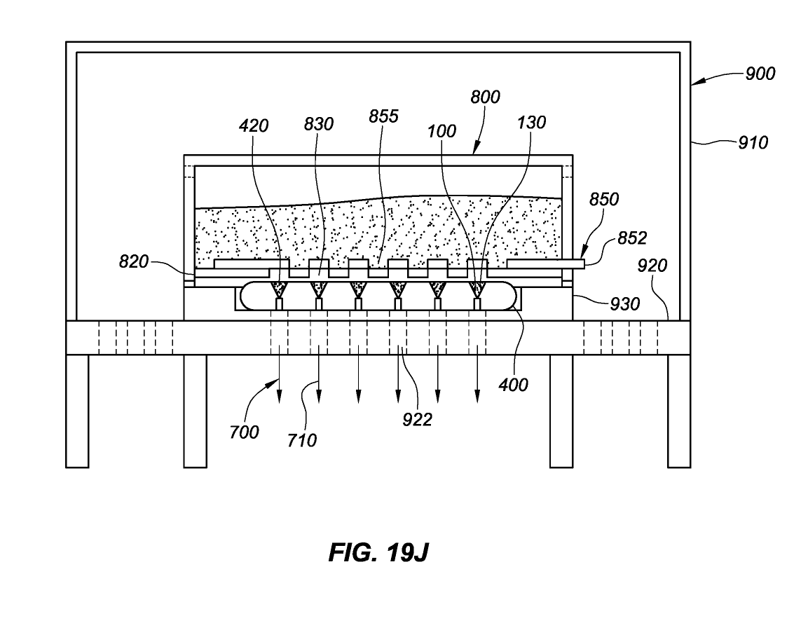

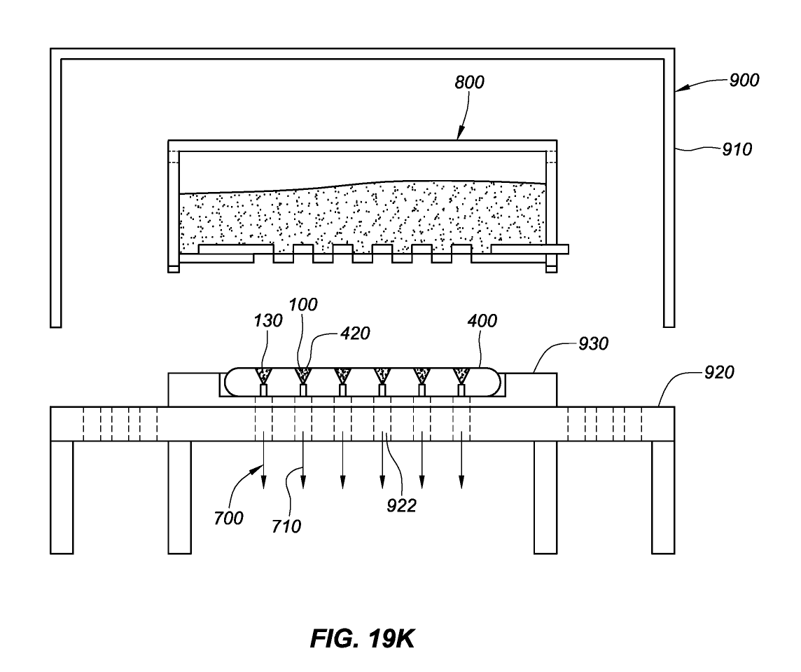

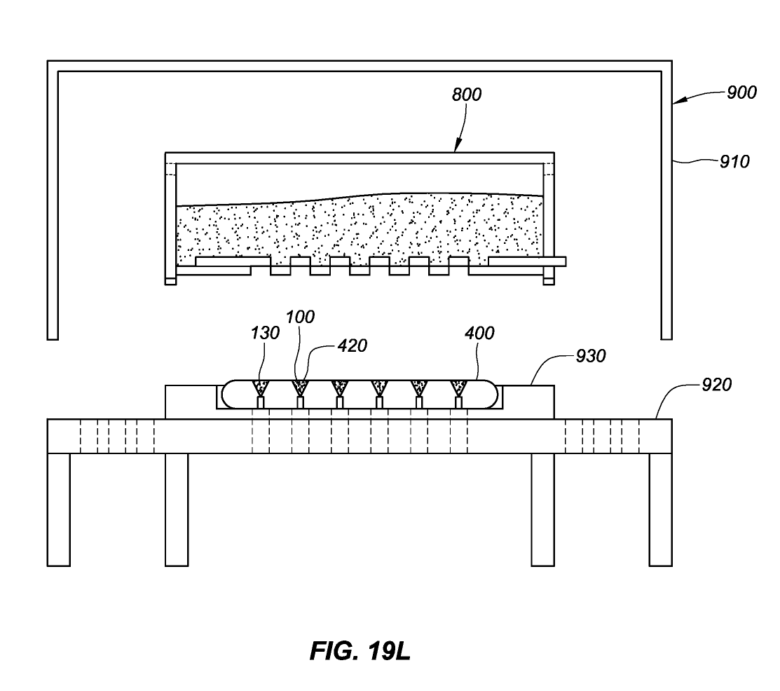

Additionally and/or alternatively, the gas-impermeable top layer can be used during a vacuum molding process. FIG. 14B illustrates another alternative embodiment of the replica mold 400. As shown in FIG. 14B, the replica mold 400 is disposed on a vacuum system 700, such as a vacuum chamber 900 (shown in FIG. 19A) and/or a vacuum table, for distributing the microneedle material 130 into the microneedle wells 420. The vacuum system 700 can be disposed adjacent to the lower region 440 of the replica mold 400 and subject the replica mold 400 to vacuum from below in order to draw the microneedle material 130 into the microneedle wells 420. Although available for activation and/or deactivation at any suitable time, the vacuum system 700 preferably is activated as the microneedle material 130 is being disposed on the replica mold 400 and can remain activated until the microneedle material 130 within the replica mold 400 is ready for drying, at 356. In other words, to increase immediacy of the suction, the vacuum system 700 can pull the vacuum on the empty replica mold 400 to degas the replica mold 400 prior to dispensing of the microneedle material 130.

The excess microneedle material 130 (i.e., the volume of microneedle material 130 in excess of that required to fill the microneedle wells 420) can remain on the upper region 410 of the replica mold 400 and eventually can form the residual layer 150. The specific vacuum pressure for filling the microneedle wells 420 can vary based on a gas permeability and thickness of the replica mold 400 and/or a viscosity of the microneedle material 130. The vacuum drawn by the vacuum system 700 preferably is sufficient for drawing the microneedle material 130 fully into the microneedle wells 420 and to evacuate a majority of the air beneath the gas-impermeable top layer, including any air in the microneedle wells 420 and/or between any backing layer(s) 220. In one embodiment, the majority of the air beneath the gas-impermeable top layer can be removed before the backing layer 220 is disposed on the microneedle material 130.

Since surface tension can become a dominant force at the microscale, breaking down or displacing trapped air through positive pressure can become increasingly difficult. Advantageously, the air-permeability of the replica mold material 450 enables the suction of the vacuum system 700 to vacate air in the microneedle wells 420 through the replica mold 400 to efficiently fill the microstructures with the microneedle material 130. In most applications, a suitable vacuum pressure can comprise a predetermined vacuum pressure, such as approximately 20 kPa, 40 kPa, 60 kPa, 80 kPa 100 kPa or even higher, and/or a predetermined range of vacuum pressures. Exemplary preselected vacuum pressure ranges can include a predetermined vacuum pressure range between 20 kPa and 100 kPa, including any vacuum pressure sub-ranges, such as a five kilopascal sub-range (i.e., between 90 kPa and 95 kPa) and/or a ten kilopascal sub-range (i.e., between 90 kPa and 100 kPa), without limitation. The vacuum may be applied to the replica mold 400 by any suitable means including, for example, filling one or more individual replica molds 400 on a vacuum system 700 that is adapted to receive a single or multiple carrier jigs. Additionally and/or alternatively, the vacuum system 700 can simultaneously accept multiple carrier jigs each adapted for holding a single replica mold 400 and/or one or more carrier jigs each adapted for holding multiple replica molds 400.

If the vacuum system 700 comprises a vacuum chamber 900 (shown in FIG. 19A), care should be taken to not apply too much vacuum pressure. Excessive vacuum pressure can cause any gases dissolved in the microneedle material 130 to expand, thereby potentially introducing imperfections into the finally-cured microneedle array 210. In one embodiment, the microneedle material 130 can be placed in a vacuum chamber condition (removal of most of the air) for a selected length of time, such as between six seconds and ten seconds.

Filling the replica mold 400 under vacuum conditions can provide significant advantages over traditional top-filling methods. These top-filling methods typically apply a great excess of microneedle solution to a microneedle mold and then force the solution into the microneedle mold via positive pressure (e.g., centrifugation, applied by rollers, weights, etc.). These methods often result in incomplete microneedle formation because the surface tension of the air increases as the cross-sections of the well recesses 422 (shown in FIGS. 5A-B) formed in the microneedle mold decrease toward a bottom end region of the well recesses. Air and any other gasses may become trapped beneath the microneedle solution at the bottom tip of the well recesses, preventing complete filling of the well recesses and resulting in a dull microneedle. At these micro-dimensions, the applied pressure may be insufficient to overcome the surface tension of the air within the well recesses and/or to force trapped air to vacate up through or around the microneedle solution. Use of the replica mold 400 formed from the gas-permeable replica mold material 450 advantageously obviates these problems because any trapped air is drawn out from the bottom of the microneedle well 420, thereby promoting more-complete filling of the microneedle well 420.

After the microneedle material 130 is distributed on the replica mold 400, the microneedle material 130 can be dried (or cured), at 356. The microneedle material 130 can be dried in any suitable manner, including via solvent (for example, water) evaporation to solid form and/or application of infrared (IR) energy. Curing, such as ultraviolet (UV) light and/or crosslinking, preferably occurs in a temperature- and/or humidity-controlled oven to control patch shape, texture, and consistency, using either static curing temperature/humidity (e.g., 40 Celsius/40-30% RH) or multistage curing (e.g., ramping from 40% RH to 35% to 30%).

During the curing, the various layers (i.e., the microneedle array 210 and/or the residual layer 150) of the microneedle device 200 bond to any additional layers that may be present. As desired one or more additional layers can be added to the microneedle device 200 after the microneedle array 210 is cured.

The microneedle material 130 can be cured at a preselected temperature (and/or within a preselected range of temperatures) for a preselected time period (and/or within a preselected range of time periods) while being subjected to a preselected relative humidity (and/or within a preselected range of relative humidities). Exemplary preselected temperature ranges can include a predetermined temperature range between 25° C. and 100° C., including any temperature sub-ranges, such as a five degree sub-range (i.e., between 40° C. and 45° C.) and/or a ten degree sub-range (i.e., between 40° C. and 50° C.), within the predetermined temperature range, without limitation. Exemplary preselected time period ranges can include a predetermined time period range between thirty minutes and five hours, including any time period sub-ranges, such as a thirty minute sub-range (i.e., between one hundred and twenty minutes and one hundred and fifty minutes) and/or a one hour sub-range (i.e., between two hours and three hours), within the preselected time period range, without limitation. Exemplary preselected relative humidity ranges can include a predetermined relative humidity range between 5% RH and 50% RH, including any relative humidity sub-ranges, such as a five percent sub-range (i.e., between 40% RH and 45% RH) and/or a ten percent sub-range (i.e., between 40% RH and 50% RH), within the preselected relative humidity range, without limitation. In one embodiment, the microneedle material 130 can be cured at 60° C. for two hours while being subjected to a relative humidity between 14% RH and 30% RH. Very preferably, the microneedle material 130 can be cured at 45° C. for three hours while being subjected to a relative humidity between 7% RH and 10% RH.

Crosslinks may be physical or chemical and intermolecular or intramolecular, and crosslinking polymers can be performed in any conventional manner. Crosslinking is the process whereby adjacent polymer chains, or adjacent sections of the same polymer chain, are linked together, preventing movement away from each other. Physical crosslinking occurs due to entanglements or other physical interaction. With chemical crosslinking, functional groups are reacted to yield chemical bonds. Such bonds can be directly between functional groups on the polymer chains or a crosslinking agent can be used to link the chains together. Such an agent could possess at least two functional groups capable of reacting with groups on the polymer chains. Crosslinking prevents polymer dissolution, but may allow a polymer system to imbibe fluid and swell to many times its original size.

In some embodiments, at least some of the microneedle material 130 can be lost during the drying, at 356. If the microneedle material 130 is dried, at 356, via solvent (for example, water) evaporation to solid form, for example, a selected amount of water volume of the microneedle material 130 can evaporate during drying. The water volume loss can result in one or more of the microneedles 100 being formed as a hollow shell of dried microneedle material 130 disposed on a periphery of the microneedle wells 420. Additional microneedle material 130 can be disposed on the replica mold 400, at 352, distributed into one or more microneedle wells 420, at 354, and/or dried, at 356, in the manner set for above, to fill the hollow shells of dried microneedle material 130 and thereby form the microneedle array 210 with solid microneedles 100. In other words, the steps of disposing the microneedle material 130 on the replica mold 400, at 352, distributing the microneedle material 130 into one or more microneedle wells 420, at 354, and/or drying the microneedle material 130, at 356, can be repeated as needed to form solid microneedles 100.

Optionally, one or more quality control measures can be performed while and/or after the microneedle array 210 is formed via the replica mold 400, at 350. The quality control measures can be performed at any suitable time, such as before, during and/or after all critical steps in the manufacturing of the microneedle device 200, at 300. The microneedle material 130, for example, can be inspected for viscosity, pH and/or dry material content. The replica mold 400 can be inspected for thickness, mold cracking and/or discoloration, which may estimate residual buildup; whereas, any backing layer 220 can be inspected for thickness, holes and/or visual quality. Additionally and/or alternatively, the jig and other tools can be inspected for wear and tear, residuals, and/or sealings. These inspections can be performed in any conventional manner, including X-ray, motion check and light scanning, dissolution, disintegration, hardness/friability, uniformity of dosage units, water content, microbial limits, sterility, particulate matter, antimicrobial preservative content, extractables functionality testing, mold leachables, osmolarity, etc.

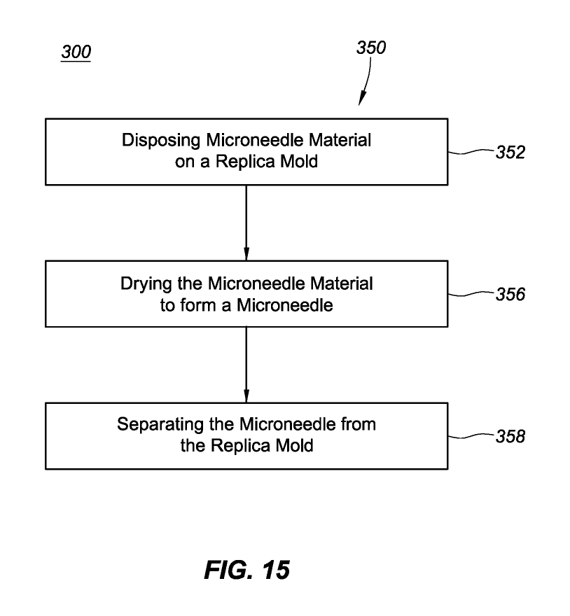

After drying of the microneedle material 130 is complete, the dried microneedle material 130 optionally can be separated from the replica mold 400, at 358, as shown in FIG. 15. The microneedle material 130 preferably is easy to release from the replica mold 400. In other words, the microneedle material 130 should be removable from the replica mold 400 without incurring damage to either the replica mold 400, the microneedle array 210 or both. If the microneedle array 210 is peeled from the replica mold 400, for example, the microneedle array 210 and/or the replica mold 400 can become folded during the peeling. The vacuum system 700 advantageously can apply a vacuum on the replica mold 400 for maintaining the shape and position of the replica mold 400 while the microneedle array 210 is being peeled from the replica mold 400. In one alternative embodiment, the replica mold 400 can serve as a storage container and/or a shipping carrier for the microneedle device 200. The microneedle array 210 thereby can be separated from the replica mold 400 by an intermediate manufacturer or an end user, reducing handling and damage to the microneedle device 200 associated with the storage and shipping process.

Manufacture of the Microneedle Array 210 with Separate Microneedles 100

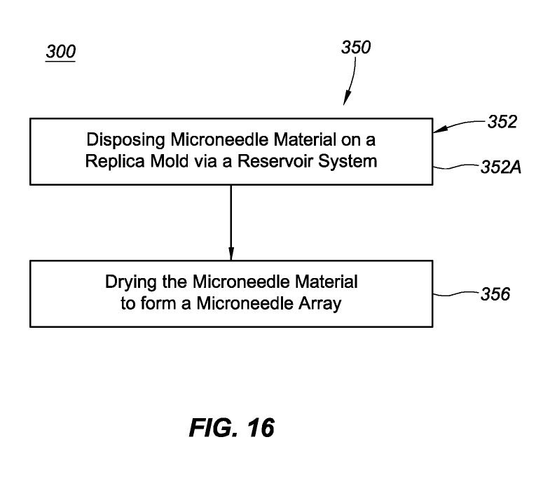

An alternative embodiment of the method 300 for manufacturing the microneedle device 200 is shown in FIG. 16. As illustrated in FIG. 16, the microneedle material 130 can be disposed onto the replica mold 400 via a reservoir system 800, at 352A, and dried (and/or cured), at 356, to form the microneedle array 210 (shown in FIGS. 2A-B, 3A). Use of the reservoir system 800 to dispose the microneedle material 130 onto the replica mold 400 advantageously can be used to manufacture the microneedle array 210 with separate microneedles 100 in the manner discussed in more detail above with reference to FIGS. 2A-B. Conventional microneedle manufacturing methods do not support disposing microneedle material 130 into individual microneedle wells 420 (shown in FIG. 14B). For example, such conventional microneedle manufacturing methods include microdroplet dispensing and microneedle manufacture by plate separation. Dispensing of microdroplets becomes quite difficult when the microneedle materials are viscous and/or elastic. Advantageously, the method 300 supports disposing microneedle material 130, including viscous and/or elastic microneedle materials 130, into the individual microneedle wells 420.

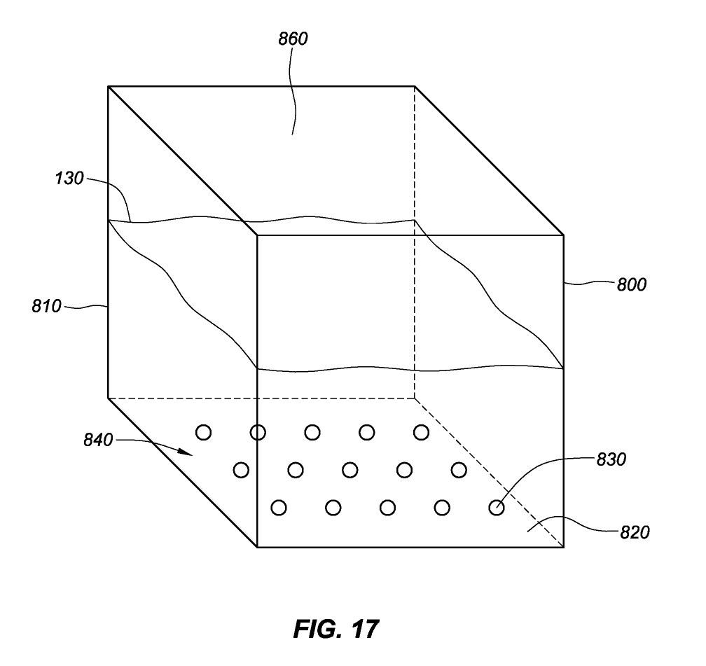

FIG. 17 illustrates an exemplary embodiment of the reservoir system 800. As shown in FIG. 17, the reservoir system 800 can comprise an enclosure (or container) 810 that defines an internal chamber 860 for receiving and/or storing a predetermined amount (or volume) of the microneedle material 130. The predetermined amount of the microneedle material 130 preferably is sufficient to fill the microneedle wells 420 (shown in FIG. 14B) formed in the replica mold 400 (shown in FIG. 14B) and can include more of the microneedle material 130 than is needed to fill the microneedle wells 420. The enclosure 810 can be constructed from any suitable material, such as a thermoplastic polymer, such as acrylic, and/or a metal, such as stainless steel, aluminum, and razor steel.

The enclosure 810 includes a lower region (or surface) 820. The lower surface 820 includes a reservoir opening array (or stencil) 840 that includes one or more reservoir openings 830 and that are in fluid communication with the internal chamber 860. The lower surface 820 preferably is substantially flat and rigid so that a liquid- and/or gas-tight seal can be made with the replica mold 400. The seal between the lower surface 820 and the replica mold 400 can help to ensure that the microneedle material 130 can flow from the reservoir system 800 directly into the microneedle wells 420 with substantially no leakage. The lower surface 820 can be formed from, or coated with, a hydrophobic material to help reduce loss of the microneedle material 130 when the reservoir system 800 is separated from the replica mold 400. The lower surface 820 likewise can have a predetermined stencil thickness. The predetermined stencil thickness can comprise any suitable thickness, such as 0.1 mm, 0.2 mm or 0.3 mm, or any suitable range of thicknesses.

The reservoir openings 830 can be arranged in any predetermined pattern. For example, the reservoir opening array 840 can include one or more reservoir openings 830 disposed in a regularly-distributed pattern and/or one or more reservoir openings 830 disposed in an irregularly-distributed (or random) pattern. An exemplary regularly-distributed pattern for the reservoir opening array 840 can comprise can include a plurality of parallel rows of the reservoir openings 830 and/or a plurality of parallel columns of the reservoir openings 830. The reservoir openings 830 preferably are arranged in a pattern that corresponds with the predetermined pattern of the microneedle wells 420 formed in the replica mold 400. In other words, each reservoir opening 830 preferably aligns with a corresponding microneedle well 420 of the replica mold 400.

The dimensions of the reservoir openings 830 formed in the reservoir system 800 can be greater than, less than and/or equal to the dimensions of the microneedle wells 420 formed in the replica mold 400, and the shapes of the reservoir openings 830 can be the same as, or different from, the shapes of the microneedle wells 420. Although shown and described as having reservoir openings 830 with uniform shape, size and/or dimension for purposes of illustration only, the reservoir opening array 840 can include reservoir openings 830 with uniform and/or different shapes, sizes and/or dimensions as desired.

One manner by which the microneedle material 130 can be disposed onto the replica mold 400 via the reservoir system 800, at 352A, and dried (and/or cured), at 356, to form the microneedle array 210 as illustrated in FIGS. 18A-E. As shown in FIG. 18A, the reservoir system 800 can receive and/or store the microneedle material 130 within the enclosure 810. The reservoir system 800 of FIG. 18A includes an optional shutter system 850 for selectively opening and/or closing the fluid communication between the internal chamber 860 and the reservoir openings 830. Stated somewhat differently, a flow of the microneedle material 130 from the enclosure 810 through the reservoir openings 830 can be controlled via the shutter system 850.

The reservoir system 800 can be positioned adjacent to, and lowered toward, the replica mold 400. The replica mold 400 is shown as being disposed on the vacuum system 700 for generating a closed vacuum as the reservoir system 800 is lowered toward the replica mold 400. The reservoir openings 830 of the reservoir opening array 840 preferably are axially aligned with the microneedle wells 420 of the microneedle well array 430. Thereby, when the reservoir system 800 and the replica mold 400 make physical contact, the reservoir openings 830 can be in fluid communication with the microneedle wells 420 as illustrated in FIG. 18B. The vacuum system 700 can maintain the closed vacuum as the reservoir system 800 is positioned on the replica mold 400. As desired, the reservoir system 800 can receive the microneedle material 130 before and/or after being positioned on the replica mold 400.

The shutter system 850 can be opened at a predetermined time as shown in FIG. 18C. Although preferably no longer maintaining the closed vacuum, the vacuum system 700 can be activated to apply suction to the replica mold 400 before, after and/or at the predetermined time. Once the shutter system 850 is opened, the suction provided by the vacuum system 700 can draw the microneedle material 130 from the enclosure 810 through the reservoir openings 830 and onto the respective microneedle wells 420 of the replica mold 400.

Once a suitable amount of the microneedle material 130 is disposed within the microneedle wells 420, the shutter system 850 can close, stopping the reservoir system 800 from dispensing any additional microneedle material 130 onto the microneedle wells 420 as shown in FIG. 18D. The reservoir system 800 then can be withdrawn (or separated) from the replica mold 400 as illustrated in FIG. 18E.

In one embodiment, the microneedle material 130 can be disposed onto the replica mold 400, at 352A (shown in FIG. 16), by placing the reservoir system 800 on top of the replica mold 400 and filling the microneedle wells 420 with the microneedle material 130. The vacuum system 700 can apply the vacuum for drawing the microneedle material 130 into, and filling, the microneedle wells 420 in a single step. Once the microneedle wells 420 have been filled with the microneedle material 130, the reservoir system 800 can be separated from the replica mold 400. Any additional layers, such as the optional backing layer 220, can be applied to the replica mold 400, and the microneedle material 130 can be cured, at 356 (shown in FIG. 16), to form the separate microneedles 100. Additionally and/or alternatively, the additional layers can be added before curing the microneedle material 130, at 356, such that a top-facing surface of the microneedles 100 can become bonded to the most proximal (or bottom) additional layers during curing of the microneedle material 130, at 356.

In an alternative embodiment, the microneedle material 130 can be disposed onto the replica mold 400, at 352A, by placing the reservoir system 800 on top of the replica mold 400 and filling the microneedle wells 420 with the microneedle material 130. The vacuum system 700 can apply the vacuum for drawing the microneedle material 130 into the microneedle wells 420. Here, the microneedle material 130 can be disposed onto the replica mold 400, at 352A, as a series of partial disposals of the microneedle material 130 under vacuum.

Each of the partial disposals of the microneedle material 130 can be disposed around an intermediate curing of the disposed microneedle material 130, at 356. In other words, the microneedle wells 420 are partially filled with a first dispensing of the microneedle material 130 from the reservoir system 800, at 352A. The first dispensing of the microneedle material 130 is cured, at 356. A second dispensing of the microneedle material 130 from the reservoir system 800 is dispensed into the microneedle wells 420, at 352A, and the dispensed microneedle material 130 in the microneedle wells 420 is cured, at 356, and so on. After each partial filling, the microneedle material 130 in the microneedle wells 420 can be partially and/or completely cured. The vacuum can maintain the vacuum or suspend the vacuum for a selected curing step, and/or the curing, at 356, can include infrared curing for removing water from the dispensed microneedle material 130. The cycle of filling, at 352A, and curing, at 356, can be repeated until the microneedle wells 420 are completely filled with the dispensed microneedle material 130.

The intermediate curing process advantageously can improve the filling of the microneedle wells 420 with the dispensed microneedle material 130 and/or can promote formation of solid microneedles 100. The microneedle material 130 can comprise approximately ninety percent water, which is lost during curing, at 356. The intermediate curing process can drive off a substantial portion of the water in the microneedle material 130, allowing for the incorporation of more microneedle polymer, such as hyaluronic acid (HA) and/or crosslinked materials, in the finally-formed microneedle 100. The intermediate curing may be done by any suitable process. For high throughput applications, the partially-formed microneedles 100 can undergo infrared (IR) curing without removing the carrier jig from the vacuum system 700.

In some embodiments, for example, the microneedles 100 are made of a material that contains hyaluronic acid, or derivative thereof, that is crosslinked with a cationic agent. In at least one embodiment, the microneedles 100 comprise hyaluronic acid, or derivative thereof, that is crosslinked with chitosan or a derivative thereof. In some embodiments, the microneedles 100 are made of a material that contains polyvinylpyrrolidone, polyvinylalcohol, a cellulose derivative, or other water soluble biocompatible polymer. In some embodiments, the microneedles 100 are made of a material that contains polyvinylpyrrolidone having an average molecular weight between about 20 kDa and about 100 kDa. In some embodiments, the substrate is made of a material that contains polyvinylpyrrolidone having an average molecular weight between about 20 kDa and about 100 kDa. In some embodiments, the substrate is made of a material comprising between about 20% and about 50% polyvinylalcohol.

In some embodiments, HA can be complexed with a suitable crosslinking agent. The crosslinking agent may be any agent known to be suitable for crosslinking polysaccharides and their derivatives via their hydroxyl groups. Suitable crosslinking agents include, but are not limited to, 1,4-butanediol diglycidyl ether (or 1,4-bis(2,3-epoxypropoxy)butane or 1,4-bisglycidyloxybutane, all of which are commonly known as BDDE), 1,2-bis(2,3-epoxypropoxy)ethylene and 1-(2,3-epoxypropyl)-2,3-epoxycyclohexane. The use of more than one crosslinking agent or a different crosslinking agent is not excluded from the scope of the present disclosure. The step of crosslinking may be carried out using any means known to those of ordinary skill in the art. Those skilled in the art appreciate how to optimize conditions of crosslinking according to the nature of the HA, and how to carry out crosslinking to an optimized degree. Degree of crosslinking for purposes of the present disclosure is defined as the percent weight ratio of the crosslinking agent to HA-monomeric units within the crosslinked portion of the HA based composition. It is measured by the weight ratio of HA monomers to crosslinker (HA monomers:crosslinker). In some embodiments, the degree of crosslinking in the HA component of the present compositions is at least about 2% and is up to about 20%. In other embodiments, the degree of crosslinking is greater than 5%, for example, is about 6% to about 8%. In some embodiments, the degree of crosslinking is between about 4% to about 12%. In some embodiments, the degree of crosslinking is less than about 6%, for example, is less than about 5%. In some embodiments, the HA component is capable of absorbing at least about one time its weight in water. When neutralized and swollen, the crosslinked HA component and water absorbed by the crosslinked HA component is in a weight ratio of about 1:1. The resulting hydrated HA-based gels have a characteristic of being highly cohesive.

In some embodiments, the polymers of the microneedles 100 are crosslinked, either physically, chemically or both and/or intermolecular or intramolecular. The microneedle array can comprise groups of microneedles 100 wherein a first group comprises at least one different cross-linker to at least a second group. Additionally and/or alternatively, the microneedles 100 may not be crosslinked and will dissolve following an initial swelling phase upon puncturing the stratum corneum and coming into contact with skin moisture. In this case, the therapeutic active agents can be released into the skin at a rate determined by the rate of dissolution of the microneedles 100.

The rate of dissolution of particular microneedles 100 is dependent on their physicochemical properties which can be tailored to suit a given application or desired rate of drug release. Relatively slow dissolution times can, in some cases, advantageously enable prolonged retention of the active compound. In some embodiments, the microneedles 100 can have a dissolution time of about or at least about 60, 75, 90, 105, 120, 135, 150, 165, 180, 195, 210, 225, 240, 300, 360, 420, 480, 600, 720, or more minutes, or 6, 8, 10, 12, 14, 16, 18, 20, 22, 24, 28, 32, 36, 40, 44, 48 hours, or more.

In some embodiments, microneedles absorb interstitial fluids, e.g., fluids within the skin in order to increase volume and provide an improved aesthetic appearance, e.g., to eliminate or improve wrinkles for example. In some embodiments, the microneedles can, after insertion into the stratum corneum, have a maximal increase in weight (e.g., by the absorption of interstitial fluid) of about or at least about 20%, 40%, 60%, 80%, 100%, 120%, 140%, 160%, 180%, 200%, 220%, 240%, 260%, 280%, 300%, 350%, 400%, 500%, 600%, 700%, 800%, 900%, 1,000%, or more. In some embodiments, the maximal increase in weight (after which the weight of the microneedles can decrease as they dissolve), occurs after about or at least about 60, 75, 90, 105, 120, 135, 150, 165, 180, 195, 210, 225, 240, 300, 360, 420, 480, 600, 720, or more minutes.

Combinations of non-crosslinked, lightly crosslinked and extensively crosslinked microneedles 100 can be combined in a single device so as to deliver a bolus dose of an active agent e.g. or therapeutic substance(s), achieving a therapeutic plasma level, followed by controlled delivery to maintain this level. This strategy can be successfully employed whether the therapeutic substance is contained in the microneedles 100 and substrate or in an attached reservoir (not shown).

Dispensing of the microneedle material 130 from the reservoir system 800 can be aided by a pressure pulse formed within the enclosure 810. For example, a positive pressure can be applied to a top surface of the microneedle material 130 within the reservoir system 800 to drive the microneedle material 130 from the reservoir system 800 and into the microneedle wells 420. In one embodiment, the pressure pulse can be provided via a pressure valve and/or a pressurized sack that can through explosion and/or implosion create the pressure pulse in the reservoir system in a controlled manner. Additionally and/or alternatively, a second vacuum can be applied to the top surface of the microneedle material 130 within the reservoir system 800. This second vacuum can supplement the suction of the vacuum system 700 below the replica mold 400. The second vacuum, for example, can be provided via the same source that provides the above-referenced pressure pulse and/or via a separately-controlled vacuum source (not shown) for controlling top surface vacuum attributes to aid degassing and controlling of the dispensed materials. Advantageously, the second vacuum can vacate air and other dissolved gases from the uncured microneedle material 130 within the reservoir system 800 and promote more complete filling of the microneedle wells 420.

After the final filling step, the microneedles 100 may be subjected to an optional intermediate cure step. In one embodiment, the microneedles 100 are not subject to an intermediate cure step after the final filling step. The reservoir system 800 can be separated from the replica mold 400 and/or additional layers, as described above, can be added to the top-facing surface of the microneedles 100. The microneedles 100 can be subject to an optional final curing, at 356, for bonding the base region 110 of the microneedles 100 to the bottom face of the additional layers to produce a unitary microneedle device 200 with any predetermined number of individual microneedles 100 in any predetermined arrangement and/or configuration and, in some embodiments, without a residual layer 130 for connecting the individual microneedles 100.

In some embodiments, the microneedle device 200 can undergo a final curing process. Typically, the final curing process can provide a more complete curing of the microneedle material 130 than the microneedle material 130 underwent during the intermediate curing step(s). Suitable final curing conditions include, for example, room temperature curing at a room temperature between 21° C.-30° C. and a relative humidity of 40% RH±10% RH for a predetermined time between two hours and five hours, or environmental cabinet curing at a cabinet temperature of about 40° C. and a relative humidity of 20% RH±10% RH, or of 40% RH 10% RH, or a combination of relative humidity, for a predetermined time between fifteen minutes and sixty minutes. Faster curing and/or higher temperature current can be achieved, for example, via a combination of curing processes, such as infrared curing and heated air curing at low relative humidity. Additionally and/or alternatively, the curing can occur within an inert gas with low humidity and/or within a disinfectant/vacuum, which would destroy bacteria and other contaminants.

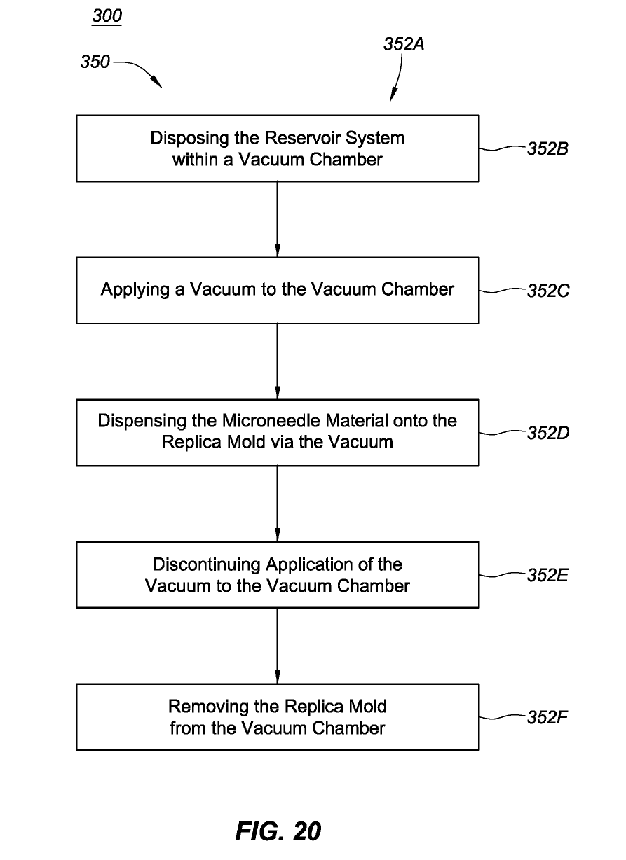

Additionally and/or alternatively, the reservoir system 800 can be disposed, at 352B, in a vacuum chamber 900 as illustrated in FIGS. 19A and 20. The vacuum chamber 900 can be provided in any conventional manner. The exemplary vacuum chamber 900 of FIG. 19A is shown as comprising a vacuum chamber cover 910 and a vacuum chamber base 920. The vacuum chamber cover 910 and/or the vacuum chamber base 920 can define a central chamber region 915 for receiving the reservoir system 800 and can be disposed in an open (or unsealed) position as shown in FIG. 19A or a closed (or sealed) position as shown in FIG. 19B. In the closed position, the vacuum chamber cover 910 can cooperate with the vacuum chamber base 920 such that the vacuum chamber cover 910 and the vacuum chamber base 920 form an air-tight bond for the central chamber region 915. As desired, the vacuum chamber cover 910 and the vacuum chamber base 920 can comprise separate vacuum chamber elements and/or can be coupled, for example, via a hinge or other coupling member (not shown).

The vacuum chamber base 920 can include a mold support region 930 for supporting the replica mold 400. The mold support region 930 preferably is centrally disposed at the vacuum chamber base 920 and can comprise a planar support and/or, as illustrated in FIG. 9A, include a support extension 935 that extends from the vacuum chamber base 920. The support extension 935 can be at least partially integrated with, and/or separate from, the vacuum chamber base 920. The mold support region 930 can receive and/or engage the replica mold 400 such that at least some of the microneedle wells 420 formed in the replica mold 400 can communicate with one or more vacuum openings 922 formed in the vacuum chamber base 920 and/or the mold support region 930. Preferably, each of the microneedle wells 420 is axially aligned with a respective vacuum opening 922 when the replica mold 400 is properly engaged by the mold support region 930.

The vacuum chamber base 920 and/or the mold support region 930 can further define one or more optional peripheral vacuum openings 924. The vacuum openings 922 and/or the peripheral vacuum openings 924 can be formed in any predetermined pattern by the vacuum chamber base 920 and/or the mold support region 930. For example, the vacuum openings 922 preferably are provided in a predetermined pattern that corresponds with the predetermined pattern of the microneedle wells 420 formed in the replica mold 400. The peripheral vacuum openings 924 can be disposed in a predetermined pattern at one or more locations about a periphery of the mold support region 930. In a preferred embodiment, the mold support region 930 and/or the vacuum openings 922 can be disposed centrally among the peripheral vacuum openings 924. Stated somewhat differently, the peripheral vacuum openings 924 preferably are formed by the vacuum chamber base 920 on each side of (or bounding) the mold support region 930 and/or the vacuum openings 922.

With the replica mold 400 engaged by the mold support region 930 and the reservoir system 800 being disposed within the central chamber region 915, the vacuum chamber 900 can transition from the open position to the closed position as shown in FIGS. 19A-B. A vacuum 710, 720 can be applied to the closed vacuum chamber 900, at 352C, as illustrated in FIGS. 19C and 20. The vacuum 710, 720 can be applied to the empty replica mold 400, for example, to degas the replica mold 400 prior to dispensing of the microneedle material 130 and/or to the microneedle material 130, for example, to degas the microneedle material 130 prior to being dispensed onto the replica mold 400. In a preferred embodiment, the vacuum 710, 720 can be applied via a vacuum system 700 in the manner discussed herein with reference to FIGS. 14B and 18A-E. The vacuum 710, 720 can include a central vacuum 710 applied via the vacuum openings 922 formed in the vacuum chamber base 920 and/or the mold support region 930 and/or a peripheral vacuum 720 applied via the optional peripheral vacuum openings 924 formed in the vacuum chamber base 920. Preferably being independently controllable, the central vacuum 710 and the peripheral vacuum 720 can be applied to the closed vacuum chamber 900 via respective vacuum systems 700, and/or a selected vacuum system 700 can at least partially provide the central vacuum 710 and the peripheral vacuum 720.

Once the applied vacuum within the central chamber region 915 of the closed vacuum chamber 900 achieves one or more predetermined criteria, the microneedle material 130 within the reservoir system 800 can be dispensed onto the replica mold 400, at 352D, as shown in FIGS. 19D-J and 20. Exemplary predetermined criteria can include the central chamber region 915 achieving a selected internal pressure level, a selected internal temperature level and/or a selected relative humidity level. Optionally, the selected internal pressure level can comprise a pressure level within a preselected range of internal pressure levels, and/or the selected internal temperature level can comprise a temperature level within a preselected range of internal temperature levels. The selected internal pressure level likewise can optionally comprise a relative humidity level within a preselected range of internal relative humidity levels. Illustrative pressure, temperature and relative humidity levels are set forth herein.

Turning to FIG. 19D, the reservoir system 800 can be positioned adjacent to the replica mold 400 within the central chamber region 915. The reservoir system 800 preferably is positioned such that the microneedle wells 420 formed in the replica mold 400 can be aligned with the reservoir openings 830 of the reservoir system 800. Very preferably, each of the microneedle wells 420 is axially aligned with a respective reservoir opening 830 when the reservoir system 800 is properly positioned. The shutter system 850 is shown as being in a closed position and thereby inhibits a flow of microneedle material 130 stored within the reservoir system 800 into the microneedle wells 420 via the reservoir openings 830.

The vacuum 710, 720 applied to the central chamber region 915 of the closed vacuum chamber 900 can comprise an adjustable vacuum. In one embodiment, the central vacuum 710 can be adjusted cooperatively with, and/or independently of, the peripheral vacuum 720. The central vacuum 710, for example, can be maintained while the peripheral vacuum 720 can be at least temporarily stopped in the manner illustrated in FIG. 19E. Control of the vacuum 710, 720 can be provided in any manual and/or automated manner.

The shutter system 850 can be transitioned from a closed position to an open position. In the open position, the shutter system 850 enables the reservoir openings 830 of the reservoir system 800 to communicate with the microneedle wells 420 formed in the replica mold 400 as shown in FIG. 19F. In other words, a reservoir opening array 840 of the reservoir system 800 can communicate with the microneedle wells 420 of the replica mold 400. The reservoir system 800 thereby can be configured to dispense microneedle material 130 onto the replica mold 400.

FIGS. 19F-H illustrate an exemplary embodiment of the shutter system 850. Turning to FIG. 19G, the shutter system 850 can comprise a shutter member 852 that defines a reservoir opening array 840 with a plurality of shutter openings 855. The shutter member 852 can slidably (or otherwise movably) engage the lower region 820 of the reservoir system 800. In other words, the shutter member 852 can move relative to the lower region 820 of the reservoir system 800. The relative motion between the shutter member 852 and the lower region 820 can include, for example, a translation and/or a rotation. The shutter member 852 can move relative to a stationary lower region 820, the lower region 820 can move relative to a stationary shutter member 852, or both the shutter member 852 and the lower region 820 can be movable.

The shutter openings 855 can be formed in the shutter member 852 with any predetermined pattern. Preferably, the shutter openings 855 are provided in a predetermined pattern that corresponds with the predetermined pattern of the reservoir openings 830 of the reservoir system 800. The shutter openings 855 preferably are not aligned with the reservoir openings 830 in the closed position as shown in FIG. 19G. The shutter member 852 thereby can obstruct any flow through the reservoir openings 830. When the shutter system 850 is actuated to transition from the closed position to the open position, the shutter openings 855 preferably are aligned with the reservoir openings 830 as illustrated in FIG. 19H. In the open position, the reservoir openings 830 are not obstructed by the shutter member 852, and flow can be provided through the aligned shutter openings 855 and reservoir openings 830.

Returning briefly to FIG. 19F, the microneedle material 130 stored within the reservoir system 800 can be permitted to flow through the reservoir openings 830 and into the microneedle wells 420 via the shutter system 850 in the open position. The flow of the microneedle material 130 into the microneedle wells 420 can be facilitated via the central vacuum 710. The central vacuum 710, for example, can help draw the microneedle material 130 from the reservoir system 800 and into the microneedle wells 420 in the manner illustrated in FIG. 19I. In a preferred embodiment, the central vacuum 710 can be adjusted to a suitable level for facilitating the flow of the microneedle material 130 into the microneedle wells 420. Exemplary adjustments can include increasing the central vacuum 710, decreasing the central vacuum 710, and at least temporarily stopping the central vacuum 710. A predetermined volume of the microneedle material 130 thereby can be disposed within the microneedle wells 420 of the replica mold 400. Each microneedle well 420 of the replica mold 400 preferably is at least ninety percent filled, such as between ninety-five percent and one hundred percent filled, with the microneedle material 130.

The shutter system 850 likewise can be actuated to transition from the open position to the closed position as illustrated in FIG. 19J. In other words, the shutter system 850 can return to the closed position. Actuation of the shutter system 850 can be triggered by any conventional manner. The vacuum chamber 900, for example, can include a control system (not shown) for actuating the shutter system 850 of the reservoir system 800. The control system can enable manual and/or automated actuation of the shutter system 850. As set forth in more detail herein, the shutter system 850 can selectively open and/or close the fluid communication between the internal chamber 860 and the reservoir openings 830 of the reservoir system 800. In other words, the control system can control a flow of microneedle material 130 stored within the internal chamber 860 through the reservoir openings 830 via the shutter system 850. The shutter system 850 thereby can be actuated while the vacuum chamber 900 is disposed in the closed position and without breaking the vacuum within the vacuum chamber 900.

Actuation of the shutter system 850 from the open position to the closed position can be triggered by any predetermined criteria. The predetermined criteria, for example, can be based upon a determination that the predetermined volume of the microneedle material 130 has been disposed within the microneedle wells 420 of the replica mold 400. In the closed position, the shutter system 850 again inhibits the flow of microneedle material 130 stored within the reservoir system 800 into the microneedle wells 420 via the reservoir openings 830 in the manner discuss in more detail above. The microneedle material 130 disposed within the microneedle wells 420 forms the microneedles 100.

As desired, the shutter system 850 can be repeatedly actuated to transition between the closed and open positions and back to the closed position multiple times. Additional microneedle material 130 thereby can be successive disposed within the microneedle wells 420 until the microneedle wells 420 receive a final predetermined volume of the microneedle material 130.

FIG. 19K shows the reservoir system 800 being removed from the replica mold 400. The reservoir system 800, stated somewhat differently, is disposed distally from the replica mold 400 within the central chamber region 915. To facilitate formation of the microneedles 100, the vacuum 710 can continue to be applied to the replica mold 400 for a predetermined time period after the reservoir system 800 has been removed from the replica mold 400. The predetermined time period can include a predetermined time period range between one minute and an hour, including any time period sub-ranges, such as a five minute sub-range (i.e., between five minutes and ten minutes) and/or a ten minute sub-range (i.e., between five minutes and fifteen minutes), within the preselected time period range, without limitation.

The vacuum chamber 900 of FIG. 19K also is shown as transitioning from the closed (or sealed) position to the open position. After the predetermined time period has expired, application of the vacuum 710, 720 to the vacuum chamber 900 can be discontinued, at 352E, as illustrated in FIGS. 19L and 20. In other words, at 352E, the vacuum system 700 can be disabled. The replica mold 400 can be removed from the vacuum chamber 900, at 352F, for subsequent processing in the manner discussed above.

The disclosed embodiments are susceptible to various modifications and alternative forms, and specific examples thereof have been shown by way of example in the drawings and are herein described in detail. It should be understood, however, that the disclosed embodiments are not to be limited to the particular forms or methods disclosed, but to the contrary, the disclosed embodiments are to cover all modifications, equivalents, and alternatives.

What is claimed is:

1. A method for manufacturing a replica mold, comprising:
disposing replica mold material on a master mold; and
curing the replica mold material at a preselected high temperature for a preselected heating time period and cooling the replica mold material for a preselected cooling time period to form the replica mold.

2. The method of claim 1, wherein said disposing the replica mold material includes forming at least one microneedle well in the replica mold material via a corresponding microneedle projection extending from the master mold.

3. The method of claim 2, wherein said forming the at least one microneedle well comprises forming a microneedle well array.

4. The method of claim 1, wherein said disposing the replica mold material comprises disposing a silicone elastomer material on the master mold.

5. The method of claim 4, wherein said disposing the silicone elastomer material comprises disposing polydimethyl siloxane (PDMS) material on the master mold.

6. The method of claim 4, wherein said disposing the silicone elastomer material comprises disposing a selected silicone elastomer material that is biocompatible, is medical grade, is in an implantable class, has a low viscosity, is translucent, is transparent, has a short curing time, has a high gas permeability, has a low elongation, has a mixing ratio of about 1:1 or has a compatibility with dispenser systems.

7. The method of claim 4, wherein said disposing the silicone elastomer material comprises disposing a selected silicone elastomer material that is opaque.

8. The method of claim 6, wherein the viscosity of the selected silicone elastomer material is between one Pascal and five Pascals or the curing time of the selected silicone elastomer material is between one and fifteen minutes when exposed to heat or ultraviolet light.

9. The method of claim 4, wherein said disposing the silicone elastomer material comprises disposing a hydrophobic silicone elastomer material on the master mold.

10. The method of claim 4, wherein said disposing the silicone elastomer material comprises disposing a hydrophilic silicone elastomer material on the master mold.

11. The method of claim 1, wherein said disposing the replica mold material comprises disposing the replica mold material on the master mold manually via a syringe or automatically via a dispenser nozzle.

12. The method of claim 1, further comprising degassing the replica mold material before said disposing the replica mold material.

13. The method of claim 1, wherein the preselected high temperature is selected from a group of temperatures consisting of 150° C., 160° C., 170° C., 180° C., 190° C. and 200° C.

14. The method of claim 1, wherein the preselected high temperature comprises a predetermined range of temperatures between 150° C. and 300° C.

15. The method of claim 1, wherein the preselected heating time period is selected from a group of times consisting of five minutes, ten minutes and fifteen minutes.

16. The method of claim 1, wherein the preselected heating time period comprises a predetermined range of times between one minute and twenty minutes.

17. The method of claim 1, wherein the preselected cooling time period is selected from a group of times consisting of one minute, five minutes and ten minutes.

18. The method of claim 1, wherein the preselected heating time period comprises a predetermined range of times between thirty seconds and ten minutes.

19. The method of claim 1, wherein said cooling the replica mold material comprises disposing the replica mold material in the master mold on a cooling block.

20. The method of claim 19, wherein said disposing the replica mold material in the master mold on the cooling block comprises sequentially disposing the replica mold material in the master mold on a series of cooling regions of the cooling block.

21. The method of claim 19, wherein said disposing the replica mold material in the master mold on the cooling block comprises sequentially disposing the replica mold material in the master mold on a series of cooling blocks.

22. The method of claim 1, further comprising forming a microneedle array via the replica mold.

23. The method of claim 22, wherein said forming the microneedle array, comprises:
disposing microneedle material on the replica mold; and
curing the microneedle material to form the microneedle array.
